SEQUENCE LISTING (4 sequences)

SEQ ID NO: 1 - DNA, 20 bp, Artificial Sequence
  Reverse primer
ccatgaagccctgagatttg

SEQ ID NO: 2 - DNA, 21 bp, Artificial Sequence
  Forward primer
ttccatcaccggtacctccta

SEQ ID NO: 3 - DNA, 20 bp, Artificial Sequence
  Reverse primer
cctgaagctgcagaaaaacc

SEQ ID NO: 4 - DNA, 20 bp, Artificial Sequence
  Forward primer
tgaacgcaacagcttatgga

(19) United States
(12) Patent Application Publication
(10) Pub. No.: US 2022/0008448 A1
(43) Pub. Date: Jan. 13, 2022

(54) METHODS OF TREATING FLT3-MUTATED HEMATOLOGIC CANCERS

(71) Applicants: CTI BIOPHARMA CORP., Seattle, WA (US); OHIO STATE INNOVATION FOUNDATION, Columbus, OH (US)

(72) Inventors: Bhavana BHATNAGAR, Columbus, OH (US); Sharyn D. BAKER, Columbus, OH (US)

(21) Appl. No.: 17/282,170

(22) PCT Filed: Oct. 1, 2019
(86) PCT No.: PCT/US2019/054142

§ 371 Date: Apr. 1, 2021

Related U.S. Application Data
(60) Provisional application No. 62/739,802, filed on Oct. 1, 2018.
(60) Provisional application No. 62/838,239, filed on Apr. 24, 2019.

(51) Int. Cl. A61K 31/7068; A61K 31/529; A61K 31/704; A61K 31/706; A61P 35/02

(52) U.S. Cl. CPC A61K 31/7068; A61K 31/529; A61P 35/02; A61K 31/706; A61K 31/704

(57) ABSTRACT

Methods to inhibit FLT3 activity in a subject or a cell with an FLT3 mutation are provided. Methods of treating a hematologic cancer, such as acute myeloid leukemia, in a subject identified as having an FLT3 mutation, are also provided.

### BACKGROUND

### Technical Field

Embodiments of the present invention are generally directed to treatment of various hematologic cancers with pacritinib, for example treatment of FLT3-mutated acute myeloid leukemia.

### Description of the Related Art

Acute myeloid leukemia (AML) is a clonal hematopoietic disorder characterized by genetic and epigenetic alterations that lead to a block in granulocyte differentiation and accumulation of leukemic blasts in blood and bone marrow (BM). Despite the adaptation of cytogenetically risk-stratified therapies, 20% to 30% of AML patients never achieve complete remission, and greater than 50% of patients who achieve complete remission subsequently experience very early disease relapse. The lack of significant advances in the treatment of AML in adults highlights the need for development of novel therapeutic strategies, particularly those directed against molecular targets that are known to be involved in disease pathogenesis.

Nonrandom chromosomal abnormalities (e.g., deletions, translocations) are identified in approximately 55% of all adult primary AML patients. Mutations in the fms-like tyrosine kinase 3 (FLT3) gene were one of the first molecular abnormalities to be described in AML. FLT3 is a type 3 receptor tyrosine kinase expressed on normal bone marrow progenitor cells; and its expression is normally lost with maturation of these progenitors. However, FLT3 is expressed on AML cells in at least seventy-percent of cases, and approximately a third of AML patients harbor activating mutations of FLT3, including internal tandem duplications (ITDs) in 25% and point mutations (TKDs) in 5%, resulting in constitutive activation of FLT3 signaling.

The presence of the FLT3 mutation has a well-recognized adverse prognostic impact on disease outcomes, with short disease-free survival following standard AML, chemotherapy. As such, a number of FLT3 inhibitors have been evaluated, initially as single agents, then in combination with chemotherapy, to assess AML response and impact on outcomes in AML patients who carry the FLT3 mutation. However, these “first-generation” FLT3 inhibitors may not be optimal due to high plasma protein binding, cell cycle inhibition, and multikinase inhibition that may result in off-target effects and toxicities, therefore leading to evaluation of these agents in combination with standard chemotherapy regimens with mixed results. Nonetheless, there remains a need in the art for effective FLT3 inhibition, including FLT3 mutants, as a therapeutic target. The present disclosure provides this and related advantages.

### BRIEF SUMMARY

Embodiments of the present invention are generally directed to methods of treating a subject or cells having a hematologic cancer with an FLT3 mutation.

In brief, some embodiments provide a method for treating cancer, the method including administering an effective amount of a therapeutic agent having inhibitory activity against Janus kinase 2 (JAK2) and fms-like tyrosine kinase 3 (FLT3) to a subject with a predetermined genetic profile comprising an FLT3 mutation. In particular embodiments, the FLT3 mutation is an internal tandem duplication mutation, and/or a tyrosine kinase domain mutation. In particular embodiments, the therapeutic agent is pacritinib, or a pharmaceutically acceptable salt or N-oxide thereof.

Some embodiments of the present invention provide methods of treating FLT3-mutated acute myeloid leukemia. One embodiment provides a method of selecting a treatment regimen and treating a subject, the method including receiving a genetic profile for the subject and treating the subject based on the genetic profile. In certain embodiments, the genetic profile is an FLT3 mutation. In particular embodiments, the FLT3 mutation is an internal tandem duplication mutation, and/or a tyrosine kinase domain mutation.

Another related embodiment provides a method of inhibiting FLT3 activity in a cell with an FLT3 mutation, the method comprising contacting the cell with an effective amount of pacritinib. In particular embodiments, the cell is a hematopoietic cell and/or an acute myeloid leukemia cell. In particular embodiments, the inhibiting FLT3 activity causes an anti-cancer effect.

### BRIEF DESCRIPTION OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements, and have been solely selected for ease of recognition in the figures.

FIG. 1 shows dose-response curves of pacritinib binding affinity to: FLT3 (wild-type), FLT3-ITD, FLT3-ITD F691L, FLT3-ITD D835V, FLT3-ITD D835H, FLT3 D835H, FLT3 D835V, and FLT3 D835Y.

FIG. 2 shows the results of kinase assays measuring the inhibitory activity of pacritinib against FLT3-ITD (left) and FLT3 D835Y (right).

FIG. 3 shows the cellular activity of pacritinib on Ba/F3 cells transfected with: GFP (+IL3), FLT3-ITD, FLT3-ITD F691L, FLT3 D835H, FLT3 835Y, FLT3-ITD D835H, and FLT3-ITD D835Y.

FIG. 4 shows the cellular activity of pacritinib on MOLM13 cells, MV411 cells, and MOLM13-Res cells.

FIG. 5 shows a western blot of lysates from pacritinib-treated Ba/F3 cells expressing FLT3-ITD F691L (left) or FLT3-ITD D835Y (right).

FIG. 6 shows the cellular activity of pacritinib and midostaurin on murine primary leukemia cells with a double knock-in of FLT3-ITD^{+/-}/IDH2-R140Q^{+/-}.

FIG. 7 shows patient demographics and baseline characteristics.

FIG. 8 shows a study schema and patient enrollment. Patients were enrolled in one of two cohorts.

FIG. 9 shows the ex vivo viability of primary blast cells obtained from patient five, patient six, patient seven, and patient nine of the study. Pacritinib IC₅₀ values were calculated for each blast sample (top, left).

FIG. 10 shows the ex vivo viability of primary bone marrow blast cells obtained from patient five, patient six, and patient nine of the study. Cells were treated with a range of doses of pacritinib or midostaurin.

FIG. 11 shows the plasma concentration of pacritinib for the five cohort A patients who received 100 mg of pacritinib twice daily. The left panel shows plasma concentrations for the samples obtained on the first day of treatment, one hour, two hours, five hours, and twenty four hours following administration. The right panel shows plasma concentrations for pretreatment samples obtained on day five, and day twenty one of the treatment cycle.

FIG. 12 shows the plasma concentration of pacritinib for the six cohort B patients who received 100 mg of pacritinib twice daily. The left panel shows plasma concentrations for the samples obtained on the first day of treatment, one hour, two hours, five hours, and twenty four hours following administration. The right panel shows plasma concentrations for pretreatment samples obtained on day five, and day twenty one of the treatment cycle.

FIG. 13 shows the plasma concentration of pacritinib for the two cohort B patients who received 200 mg of pacritinib twice daily. The left panel shows plasma concentrations for the samples obtained on the first day of treatment, one hour, two hours, five hours, and twenty four hours following administration, and for pretreatment samples obtained on day five of the treatment cycle.

FIG. 14 provides the clinical course profile of patient five, who was enrolled in cohort A. The dose schedule is shown above the graph: pacritinib on days 1-21, ara-C (also known as cytarabine) on days 5-11, and daunorubicin on days 5-7. The graph shows platelet counts (left y axis) and white blood cell counts (right y axis), as well as the percent blasts present in bone marrow aspirates (checkered bar) and the percent blasts present in peripheral blood samples (black bar).

FIG. 15 provides the clinical course profile of patient nine, who was enrolled in cohort A. The dose schedule is shown above the graph: pacritinib on days 1-21, ara-C (also known as cytarabine) on days 5-11, and daunorubicin on days 5-7. The graph shows platelet counts (left y axis) and white blood cell counts (right y axis), as well as the percent blasts present in bone marrow aspirates (checkered bar) and the percent blasts present in peripheral blood samples (black bar).

FIG. 16 shows a Kaplan-Meier analysis for overall survival of patients who received pacritinib during the study.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense (i.e., as “including, but not limited to”).

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5% or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an”, and “the” include plural references unless the context clearly dictates otherwise.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients thereof.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid (e.g., L-(+)-tartaric acid), thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“N-oxide” refers to N⁺—O⁻, where all valences of the N atom are satisfied by bonds to the remainder of the molecule.

“Pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “effective amount” or “therapeutically effective amount” refers to that amount of compound (e.g., a compound of Formula (I)) described herein that is sufficient to effect the intended application including disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder or medical condition including a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

A “chemotherapeutic agent” refers to any agent useful for selectively killing or blocking the division of malignant cells. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

“7+3” or “7+3” treatment may refer to chemotherapy course that includes 7 days of cytarabine administeration, and 3 days of an anthracycline antibiotic or an anthracenedione, such as daunorubicin.

“Radiation therapy” means exposing a subject, using routine methods and compositions known to the practitioner, to radiation emitters such as alpha-particle emitting radionuclides (e.g., actinium and thorium radionuclides), low linear energy transfer (LET) radiation emitters (i.e., beta emitters), conversion electron emitters (e.g., strontium-89 and samarium-153-EDTMP, or high-energy radiation, including x-rays, gamma rays, and neutrons. Exemplary radiation therapies include external-beam therapy, internal radiation therapy, implant radiation, stereotactic radiosurgery, systemic radiation therapy, radiotherapy and permanent or temporary interstitial brachytherapy. Suitable radiation sources for use as a cell conditioner of the present invention include both solids and liquids. By way of non-limiting example, the radiation source can be a radionuclide, such as I¹²⁵, I¹³¹, Yb¹⁶⁹, Ir¹⁹² as a solid source, I¹²⁵ as a solid source, or other radionuclides that emit photons, beta particles, gamma radiation, or other therapeutic rays. The radioactive material can also be a fluid made from any solution of radionuclide(s), e.g., a solution of I¹²⁵ or I¹³¹, or a radioactive fluid can be produced using a slurry of a suitable fluid containing small particles of solid radionuclides, such as Au¹⁹⁸, Y⁹⁰. Moreover, the radionuclide(s) can be embodied in a gel or radioactive micro-spheres.

“Remission” may refer to a partial or complete loss of signs and/or symptoms of cancer. Types of remission include: morphologic complete remission, morphologic leukemia-free state, cytogenetic complete remission, or complete remission with incomplete hematologic recovery. Remission of AML may be defined as: <5% blasts in bone marrows aspirate, greater than or equal to 1,000 neutrophils per microliter of blood sample, greater than or equal to 100,000 platelets per microliter of blood sample, no extramedullary disease, or a combination thereof. In particular embodiments, remission may be defined as <5% blasts in bone marrow aspirates.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

The term “in vivo” refers to an event that takes place in a subject's body.

The term “in vitro” refers to an event that takes place outside a subject's body.

The term “gene” can include not only coding sequences but also regulatory regions such as promoters, enhancers, and termination regions. The term further can include all introns and other DNA sequences spliced from the mRNA transcript, along with variants resulting from alternative splice sites. Gene sequences encoding the particular protein can be DNA or RNA that directs the expression of the particular protein. These nucleic acid sequences may be a DNA strand sequence that is transcribed into RNA or an RNA sequence that is translated into the particular protein. The nucleic acid sequences include both the full-length nucleic acid sequences as well as non-full-length sequences derived from the full-length protein.

“FLT3” may refer to the gene encoding the fms-like tyrosine kinase 3 (FLT3) enzyme, and/or may refer to the encoded enzyme (UniProt ID: P36888. The FLT3 gene (NCBI Reference Sequence ID: NP_004110.2) is present on chromosome 13q12. FLT3 is expressed by bone marrow progenitor cells, but expression is normally lost during maturation of the progenitors. However, FLT3 is often aberrantly expressed on acute myeloid leukemia cells. Furthermore, certain mutations to FLT3 can cause constitutive activation of the enzyme, and activating mutations to FLT3 have been associated with cancer cell phenotypes such as enhance proliferation and survival. Approximately one-third of acute myeloid leukemia patients have mutations to the FLT3 gene, and about 25% of these are internal tandem duplications and about 5% are point mutations, such as mutation so a tyrosine kinase domain.

“JAK2” refers to Janus kinase 2 (UniProt ID: O60674), which is a non-receptor tyrosine kinase. JAK2, along with Signal Transducer and Activator of Transcription proteins (STATs), are involved in the JAK/STAT pathway, which regulates many cellular processes such as immunity and proliferation. Aberrant activation of the JAK/STAT pathway is present in many hematologic cancers, such as acute myeloid leukemia. For example a V617F mutation to JAK2 is constitutively activating and is associated with hematological cancers. Without being bound by theory, treating a subject having an FLT3 mutation with a therapeutic agent having inhibitory activity against FLT3 and JAK2 may be useful, for example, to prevent JAK2/STAT pathway-driven drug resistance.

“Internal tandem Duplication mutation” (“ITD mutation”) may refer to an FLT3 gene mutation that causes a sequence of amino acids to be duplicated in tandem within the juxtamembrane domain of FLT3. The juxtamembrane domain of FLT3 is positioned between the transmembrane domain and the first tyrosine kinase domain of the FLT3 gene, at amino acids 569-610 of FLT3. In certain embodiments, the ITD mutation can be, for example, a tandem duplication of at least three nucleotides or as many as 1500 nucleotides, either fully contained within, or at least partially overlapping the juxtamembrane domain. Exemplary methods of detecting internal tandem duplications in FLT3 can be found, for example, in Spencer et al., J Mol Diagn. 2013 January; 15(1):81-93. A compound of Formula (I), such as pacritinib, may be useful for treating ITD+ AML, for example, because it may decrease the likelihood of intrinsic and acquired drug resistance, such as the emergence of secondary tyrosine kinase domain mutations (TKD).

A “tyrosine kinase domain mutation,” or “TKD mutation” may refer to a mutation within the tyrosine kinase domain 1 (encoding AAs 610-710) or tyrosine kinase domain 2 (encoding AAs 778-943) of the FLT3 gene. TKD mutation may cause constitutive autophosphorylation and activation of FLT3. Examples of TKD mutations that have been characterized include non-synonymous mutations to: A680, F691, D835, and I836, S840, N841, and Y842 (see Bacher et al., Blood 2008 111:2527-2537; and Nguyen, et al., Oncotarget. 2017 Feb. 14; 8(7): 10931-10944). Examples of specific TKD mutations to these sites include D835Y, D835H, D835V, D835E, D835A, D835S, D835N, and Δ836, I836S, I836L, I836T, S840G. As reported by Nguyen et al., many tyrosine kinase inhibitors are not effective against a variety of TKD mutants (Nguyen, et al., Oncotarget. 2017 Feb. 14; 8(7): 10931-10944). Surprisingly, as shown in the Examples herein, Pacritinib effectively binds and inhibits a variety of TKD mutants. In certain embodiments, the TKD mutation is a non-synonymous substitution mutation, or a non-frameshift indel in a tyrosine kinase domain of FLT3. In particular embodiments, the mutation is in exon 17 and/or exon 20. TKD mutations can be identified, for example, by deep amplicon sequencing, or another sequencing method known in the art.

### Therapeutic Agents

Various embodiments provide methods including administering an effective amount of a therapeutic agent. In some embodiments, the therapeutic agent is an agent having inhibitory activity against Janus kinase 2 (JAK2) and fms-like tyrosine kinase 3 (FLT3). Without being bound by theory, an agent with inhibitory activity against JAK2 and FLT3 may be useful for treating an FLT3-mutated cancer, for example, because it may decrease the likelihood of intrinsic and acquired drug resistance such as the activation of an alternative signaling pathway (e.g., JAK/STAT).

In particular embodiments, the therapeutic agent is a compound of Formula (I) having the structure:

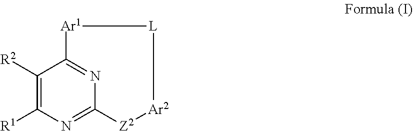

wherein:

R¹ and R² are H;

Z² is —N(H)—;

Ar¹ is selected from the group consisting of:

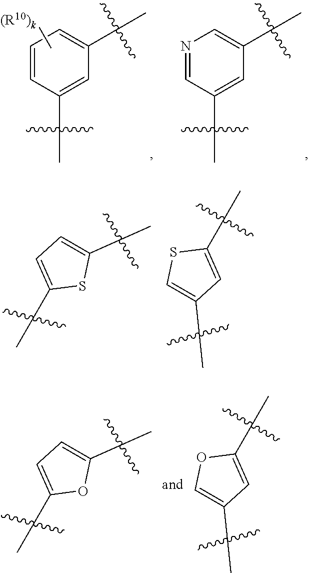

wherein R¹⁰ is methoxy or fluorine;

k is an integer selected from 0 or 1;

Ar² is a group of the formula

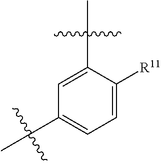

wherein R¹¹ is H or selected from the group consisting of:

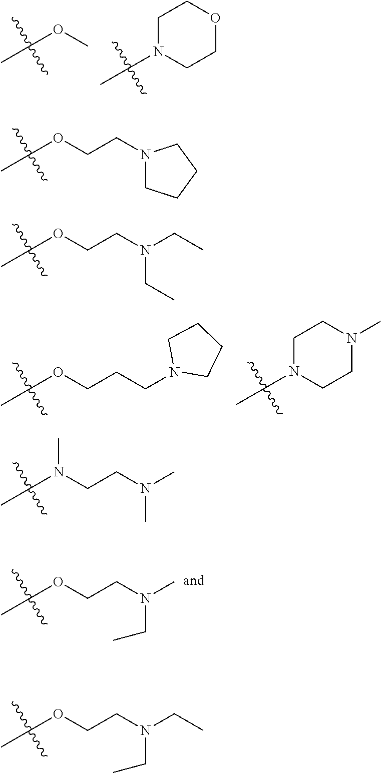

L is a group of formula:

—X¹—Y—X²—

wherein X¹ is attached to Ar¹ and X² is attached to Ar², and wherein X¹, X² and Y are selected such that the group L has between 5 and 15 atoms in the normal chain,

wherein X¹ is selected from the group consisting of:

(a) —OCH₂—

(b) —OCH₂CH₂—, and

(c) —CH₂OCH₂—;

wherein X² is selected from the group consisting of:

(a) —CH₂O—,

(b) —CH₂CH₂O—, and

(c) —CH₂OCH₂—;

Y is a group of formula —CR^{a}═CR^{b}—,

wherein R^{a} and R^{b} are H,

or a pharmaceutically acceptable salt or N-oxide thereof. In some embodiments the compound of Formula (I) is 11-(2-Pyrrolidinl-yl-ethoxy)-14,19-dioxa-5,7,26-triazatetracyclo[19.3.1.1^{2,6}.1^{8,12}] heptacosa-1(25),2(26),3,5,8,10,12(27),16,21,23-decaene (pacritinib) or a pharmaceutically acceptable salt or N-oxide thereof, such as its citrate or maleate salts. In certain embodiments, the compound of Formula (I) is 9E-15-(2-pyrrolidin-1-yl-ethoxy)-7,12,25-trioxa-19,21,24-triaza-tetracyclo[18.3.1.1(2,5).1(14,18)]hexacosa-1(24),2,4,9,14,16,18(26),20,22-nonaene, or a pharmaceutically acceptable salt or N-oxide thereof, such as its citrate or maleate salts.

### Pharmaceutical Compositions

Other embodiments are directed to pharmaceutical compositions. The pharmaceutical composition comprises a compound of Formula (I) and a pharmaceutically acceptable carrier, diluent or excipient. In some embodiments the pharmaceutical composition comprises a pharmaceutically acceptable salt thereof and a pharmaceutically acceptable carrier or excipient.

In some embodiments, the pharmaceutical composition is formulated for oral administration. For example, in some embodiments, the pharmaceutical composition comprises an oral capsule. In other embodiments, the pharmaceutical composition is formulated for injection. In some more specific embodiments, the carrier or excipient is selected from the group consisting of cellulose, lactose, carboxymethylcellulose and magnesium stearate.

In still more embodiments, the pharmaceutical compositions comprise a compound of Formula (I) or a pharmaceutically acceptable salt thereof and an additional therapeutic agent (e.g., chemotherapeutic agent). Non-limiting examples of such additional therapeutic agents are described above.

Suitable routes of administration include oral, intravenous, rectal, aerosol, parenteral, ophthalmic, pulmonary, transmucosal, transdermal, vaginal, otic, nasal, and topical administration. In addition, by way of example, parenteral delivery includes intramuscular, subcutaneous, intravenous, intramedullary injections, as well as intrathecal, direct intraventricular, intraperitoneal, intralymphatic, and intranasal injections. In particular embodiments, the compound of Formula (I) is administered orally.

The compound of Formula (I) or pharmaceutically acceptable salt thereof according to certain embodiments is effective over a wide dosage range. For example, in the treatment of adult humans, dosages from 0.01 to 2000 mg, from 1 to 1000 mg per day, from 50 to 500 mg per day, and from 200 to 400 mg per day are examples of dosages that are used in some embodiments. An exemplary dosage is between about 50 and about 500 mg per day, or is about 200 mg per day. In various embodiments, the dosage is 50, 100, 150, 200, 250, 300, 350, 400, 450, or 500 mg per day. The exact dosage will depend upon the route of administration, the form in which the compound of Formula (I) or pharmaceutically acceptable salt thereof is administered, the subject to be treated, the body weight of the subject to be treated, and the preference and experience of the attending physician.

In some embodiments, the compound of Formula (I) or pharmaceutically acceptable salt thereof is administered in a single dose. Such administration may be by injection, e.g., intravenous injection, in order to introduce the agent quickly. However, other routes are used as appropriate. A single dose of the compound of Formula (I) or pharmaceutically acceptable salt thereof may also be used for treatment of an acute condition.

In some embodiments, the compound of Formula (I) or pharmaceutically acceptable salt thereof is administered in multiple doses. In some embodiments, dosing is about once, twice, three times, four times, five times, six times, or more than six times per day. In other embodiments, dosing is about once a month, once every two weeks, once a week, or once every other day. In another embodiment the compound of Formula (I) or pharmaceutically acceptable salt thereof is administered about once per day to about four times per day. In certain embodiments, the compound of Formula (I) and the additional therapeutic agent are administered separately. In another embodiment the administration of the compound of Formula (I) or pharmaceutically acceptable salt thereof continues for less than about one month, less than about three weeks, or less than about two weeks. In certain embodiments the additional therapeutic agent is administered for less than about a month, less than about three weeks, less than about two weeks, or less than about one week. In yet another embodiment the administration of the compound of Formula (I) continues for more than about 6, 10, 14, 28 days, two months, six months, or one year. In some cases, continuous dosing is achieved and maintained as long as necessary.

Administration of the compound of Formula (I) or pharmaceutically acceptable salt thereof may continue as long as necessary or advisable. In some embodiments, the compound of Formula (I) or pharmaceutically acceptable salt thereof is administered for more than 1, 2, 3, 4, 5, 6, 7, 14, or 28 days. In some embodiments, the compound of Formula (I) or pharmaceutically acceptable salt thereof is administered for less than 28, 14, 7, 6, 5, 4, 3, 2, or 1 day. In some embodiments, the compound of Formula (I) or pharmaceutically acceptable salt thereof is administered chronically on an ongoing basis, e.g., for the treatment of chronic effects. In particular embodiments, the compound of Formula (I) may be administered daily (e.g., twice daily) on days 1-21 of a treatment cycle (e.g., a 21- or 28-day treatment cycle); on days 8-21 of a treatment cycle (e.g., a 21- or 28-day treatment cycle); or on days 5-28 of a 28 day treatment cycle.

In some embodiments, the compound of Formula (I) or pharmaceutically acceptable salt thereof is administered in dosages. It is known in the art that due to intersubj ect variability in compound pharmacokinetics, individualization of dosing regimen is necessary for optimal therapy. Dosing for the compound of Formula (I) or pharmaceutically acceptable salt thereof may be found by routine experimentation in light of the instant disclosure.

In some embodiments, the compound of Formula (I) or pharmaceutically acceptable salt thereof is formulated into pharmaceutical compositions. In specific embodiments, pharmaceutical compositions are formulated in a conventional manner using one or more physiologically acceptable carriers comprising excipients and/or auxiliaries which facilitate processing of the active compounds into preparations which can be used pharmaceutically. Proper formulation is dependent upon the route of administration chosen. Any pharmaceutically acceptable techniques, carriers, diluents and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

In certain embodiments, the compound of Formula (I) or pharmaceutically acceptable salt thereof described are administered as pharmaceutical compositions in which the compound of Formula (I) or pharmaceutically acceptable salt thereof is mixed with other active ingredients (e.g., additional therapeutic agents), as in combination therapy. Encompassed herein are all combinations of actives set forth in the combination therapies section below and throughout this disclosure.

A pharmaceutical composition, as used herein, refers to a mixture of the compound of Formula (I) or pharmaceutically acceptable salt thereof with other chemical components, such as carriers, stabilizers, diluents, dispersing agents, suspending agents, thickening agents, and/or excipients. In certain embodiments, the pharmaceutical composition facilitates administration of the compound of Formula (I) or pharmaceutically acceptable salt thereof to a subject. In some embodiments, practicing the methods of treatment or use provided herein, therapeutically effective amounts of the compound of Formula (I) or pharmaceutically acceptable salt thereof provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compound of Formula (I) or pharmaceutically acceptable salt thereof described herein are used singly or in combination with one or more therapeutic agents as components of mixtures.

In some embodiments, the subject (e.g., a human) is older than 18 years old. In some embodiments, the subject (e.g., a human) has a life expectancy of ≥3 months or ≥6 months, is not pregnant or cannot become pregnant, have acceptable liver function (e.g., bilirubin≤2.0 mg/dL, unless due to Gilbert's disease), have acceptable renal function (e.g., calculated creatine clearance≥50 mL/minute), have acceptable heart function (NYHA congestive heart failure class II or better and/or cardiac ejection fraction (LVEF)≥50%), or any combination thereof.

In some embodiments, the compound of Formula (I) or pharmaceutically acceptable salt thereof described herein is formulated for oral administration. Compounds described herein are formulated by combining the active compounds (i.e., a compound of Formula (I) or a pharmaceutically acceptable salt thereof and, optionally, additional therapeutic agents) with, e.g., pharmaceutically acceptable carriers or excipients. In various embodiments, the compound of Formula (I) or pharmaceutically acceptable salt thereof described herein are formulated in oral dosage forms that include, by way of example, tablets, powders, pills, dragees, capsules, liquids, gels, syrups, elixirs, slurries, suspensions and the like.

In certain embodiments, pharmaceutical preparations for oral use are obtained by mixing one or more solid excipient, with the compound of Formula (I) or pharmaceutically acceptable salt thereof described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses (i.e., a compound of Formula (I) or a pharmaceutically acceptable salt thereof and, optionally, additional therapeutic agents).

In certain embodiments, therapeutically effective amounts of the compound of Formula (I) or pharmaceutically acceptable salt thereof described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example, lactose, binders such as starches, and/or lubricants such as talc or magnesium stearate and, optionally, stabilizers. In other embodiments, soft capsules contain the compound of Formula (I) or pharmaceutically acceptable salt thereof that is dissolved or suspended in a suitable liquid. Suitable liquids include, by way of example, one or more fatty oil, liquid paraffin, or liquid polyethylene glycol. In addition, stabilizers are optionally added.

In other embodiments, therapeutically effective amounts of the compound of Formula (I) or pharmaceutically acceptable salt thereof described herein are formulated for buccal or sublingual administration. Formulations suitable for buccal or sublingual administration include, by way of example, tablets, lozenges, or gels. In still other embodiments, the compound of Formula (I) or pharmaceutically acceptable salt thereof described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the compound of Formula (I) or pharmaceutically acceptable salt thereof are prepared as appropriate oily injection suspensions. Suitable lipophilic solvents or vehicles for use in the pharmaceutical compositions described herein include, by way of example, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient(s) (i.e., a compound of Formula (I) or a pharmaceutically acceptable salt thereof and, optionally, additional therapeutic agents) is in powder form for constitution with a suitable vehicle, e.g., sterile pyrogen-free water, before use.

In certain embodiments, pharmaceutical compositions are formulated in any conventional manner using one or more physiologically acceptable carriers, diluents or excipients which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising the compound of Formula (I) or pharmaceutically acceptable salt thereof are manufactured in a conventional manner, such as, by way of example, by means of conventional mixing, dissolving, granulating, dragee-making, levigating, emulsifying, encapsulating, entrapping or compression processes.

Additionally, the compound of Formula (I) or pharmaceutically acceptable salt thereof described herein encompasses unsolvated as well as solvated forms with pharmaceutically acceptable solvents such as water, ethanol, and the like. The solvated forms of the compound of Formula (I) or pharmaceutically acceptable salt thereof presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compound of Formula (I) or pharmaceutically acceptable salt thereof described herein include formulating the compound of Formula (I) or pharmaceutically acceptable salt thereof with one or more inert, pharmaceutically acceptable carriers, diluents or excipients to form a solid, semi-solid or liquid. Solid compositions include powders, tablets, dispersible granules, capsules, cachets, and suppositories. Liquid compositions include solutions in which the compound of Formula (I) or pharmaceutically acceptable salt thereof is dissolved, emulsions comprising a compound, or a solution containing liposomes, micelles, or nanoparticles comprising the compound of Formula (I) or pharmaceutically acceptable salt thereof as disclosed herein. Semi-solid compositions include gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, the pharmaceutical composition comprising the compound of Formula (I) or pharmaceutically acceptable salt thereof illustratively takes the form of a liquid where the agents are present in solution, in suspension or both. Typically, when the composition is administered as a solution or suspension a first portion of the therapeutic agent (e.g., a compound of Formula (I)) is present in solution and a second portion of the therapeutic agent (e.g., a compound of Formula (I)) is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of the compound of Formula (I) or pharmaceutically acceptable salt thereof. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other compositions include one or more antioxidants to enhance chemical stability where required. Suitable antioxidants include, by way of example, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) about 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of the compound of Formula (I) or pharmaceutically acceptable salt thereof provided in the pharmaceutical compositions is less than about 100%, about 90%, about 80%, about 70%, about 60%, about 50%, about 40%, about 30%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001% w/w, w/v or v/v.

In some embodiments, the concentration of the compound of Formula (I) or pharmaceutically acceptable salt thereof is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of the compound of Formula (I) or pharmaceutically acceptable salt thereof is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of the compound of Formula (I) or pharmaceutically acceptable salt thereof is equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

### Methods

Certain methods disclosed herein provide a method of treating a subject having a predetermined mutation, and/or a method for selecting treatment regimens and methods of treatment based on a genetic mutation profile. That is, this disclosure provides methods for selecting treatment regimens as well as methods of treatment.

A first embodiment of the present invention provides a method of treating a hematologic cancer, the method comprising: administering an effective amount of a therapeutic agent having inhibitory activity against Janus kinase 2 (JAK2) and fms-like tyrosine kinase 3 (FLT3) to a subject having a predetermined genetic profile comprising an FLT3 mutation.

Such genetic profiles may be produced in any suitable manner (e.g., microarrays, reverse transcription polymerase chain reaction (RT-PCR), etc.).

In some aspects of the first embodiment, the FLT3 mutation is present in at least a subset of cells (e.g., at least 0.1% or at least 1% of cells, such as blood sample cells or bone marrow primary blast cells) present in the sample.

In some aspects of the first embodiment, the FLT3 mutation is an internal tandem duplication (ITD) mutation. The ITD mutation may be any tandem duplication within the juxtamembrane domain of FLT3.

In certain aspects of the first embodiment, the activating FLT3 mutation is a tyrosine kinase domain (TKD) mutation. In particular embodiments, the TKD mutation is an FLT 835 mutation, such as D835H, D835V, or D835Y. In particular embodiments, the TKD mutation comprises an FLT3 691 mutation, such as F691L.

In some aspects of the first embodiment, the FLT3 mutation is an ITD mutation and a TKD mutation, such as ITD-835H, ITD-835V, ITD-D835Y, or ITD-F691L.

The methods may include administering a therapeutic agent having inhibitory activity against JAK2 and FLT3. In certain embodiments, the therapeutic agent is pacritinib, or a pharmaceutically acceptable salt, prodrug, or N-oxide thereof. In particular embodiments, the therapeutic agent is a citrate salt or a maleate salt of pacritinib.

Certain aspects of the first embodiment include administering an effective amount of pacritinib. Pacritinib may be effective over a wide dosage range. In certain embodiments, the effective amount ranges from 0.01 to 2000 mg, from 1 to 1000 mg per day, from 50 to 500 mg per day, or from 200 to 400 mg per day. In particular embodiments, the effective amount ranges from between about 50 mg per day to about 500 mg per day, or is about 200 mg per day. In various embodiments, the effective amount is 50, 100, 150, 200, 250, 300, 350, 400, 450, or 500 mg per day. In particular embodiments, the effective amount is about 200 mg per day. In particular embodiments, the effective amount is about 400 mg per day.

Some aspects of the first embodiment include administering one or more further therapeutic agents, such as a chemotherapeutic agents, therapeutic antibodies, and radiation treatment, to provide a synergistic or additive therapeutic effect.

Certain aspects of the first embodiment include administering an effective amount of one or more chemotherapeutic agents. Many chemotherapeutic agents are presently known in the art and can be used in combination with a therapeutic agent having inhibitory activity against JAK2 and FLT3. In some embodiments, the chemotherapeutic agent is selected from the group consisting of mitotic inhibitors, alkylating agents, anti-metabolites (e.g., nucleoside analogs), intercalating agents, growth factor inhibitors, cell cycle inhibitors, enzymes, topoisomeRASe inhibitors, biological response modifiers, anti-hormones, angiogenesis inhibitors, hypomethylating agents, and anti-androgens.

Non-limiting examples of therapeutic agents that can be used in aspects of the first embodiment in combination with a therapeutic agent having inhibitory activity against JAK2 and FLT3 are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above.

In some aspects of the first embodiment, the one or more further therapeutic agents comprises a nucleoside analog or an intercalating agent. In other embodiments, the one or more further therapeutic agents comprise a nucleoside analog and an intercalating agent.

Nucleoside analogs function by disrupting DNA or RNA synthesis, and include purine analogs and pyrimidine analogs. In embodiments, the nucleoside analog may be: a purine analog such as fludarabine, 6-mercaptopurine, thiamiprine, thioguanine; or a pyrimidine analog, such as ancitabine, azacitidine, 6-azauridine, carmofur, cytarabine (e.g., Ara-C, dideoxyuridine, doxifluridine, enocitabine, or floxuridine. In particular embodiments, the nucleoside analog comprises cytarabine.

Intercalating agents are molecules that can bind between base pairs of DNA. In embodiments, the intercalating agent is doxorubicin, daunorubicin (also known as daunomycin), or dactinomycin. In particular embodiments, the intercalating agent is daunorubicin.

In some aspects of the first embodiment, the one or more further therapeutic agents comprises a hypomethylating agent. Hypomethylating agents are molecules that inhibit DNA methylation, for example, by inhibiting DNA methyltransferase. In particular embodiments, the hypomethylating agent comprises decitabine (also known as 5-aza-2′-deoxycytidine) or azacytidine. Decitabine may be given, for example, to a patient with relapsed or refractory AML and/or to patients who are ineligible for a more intensive therapy regimen. Decitabine may be administered, for example, at 20 mg/m², four times every twenty four hours, on days 1-10 of a 28 day treatment cycle.

When used in combination with one or more further therapeutic agents, a therapeutic agent having inhibitory activity against JAK2 and FLT3 is administered with the second agent simultaneously or separately. This administration in combination can include simultaneous administration of the two agents in the same dosage form, simultaneous administration in separate dosage forms, and separate administration. In some embodiments, a therapeutic agent having inhibitory activity against JAK2 and FLT3, and any of the agents described above (e.g., cytarabine, daunorubicin, and/or decitabine) can be simultaneously administered, wherein both the agents are present in separate formulations. In another alternative, a therapeutic agent having inhibitory activity against JAK2 and FLT3, can be administered just followed by any of the agents described above, or vice versa. In some embodiments of the separate administration protocol a therapeutic agent having inhibitory activity against JAK2 and FLT3, and any of the agents described above are administered a few minutes apart, or a few hours apart, or a few days apart.

Certain aspects of the first embodiment described herein may include treating a hematologic cancer, such as multiple myeloma, myelodysplastic syndrome (MDS), acute myeloid leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma. In some specific embodiments, the hematologic cancer is CLL, SLL, or both. In some specific embodiments, the hematologic cancer is acute myeloid leukemia (AML). In particular embodiments, the AML may be newly diagnosed AML, or relapsed or refractory AML (i.e., AML that does not undergo remission following an initial treatment). In certain specific embodiments, the acute myeloid leukemia is relapsed or refractory acute myeloid leukemia.

A second embodiment of the present invention provides a method of treating acute myeloid leukemia, the method comprising: administering an effective amount of pacritinib, or a pharmaceutically acceptable salt, prodrug, or N-oxide thereof to the subject having a predetermined mutation in FLT3, the mutation comprising: (i) an internal tandem duplication (ITD) mutation; and/or (ii) a tyrosine kinase domain (TKD) mutation.

A predetermined mutation may be identified by assaying a sample from subject using any suitable manner (e.g., microarrays, reverse transcription polymerase chain reaction (RT-PCR), etc.). In certain embodiments, the predetermined mutation is identified in a sample that was obtained from the subject prior to the treating.

In some aspects of the second embodiment, the FLT3 mutation is present in at least a subset of cells (e.g., at least 0.1% or at least 1% of cells, such as blood sample cells or bone marrow primary blast cells) present in the sample.

In some aspects of the second embodiment, the FLT3 mutation is an internal tandem duplication (ITD) mutation. The ITD mutation may be any tandem duplication within the juxtamembrane domain of FLT3.

In certain aspects of the second embodiment, the activating FLT3 mutation is a tyrosine kinase domain (TKD) mutation. In particular embodiments, the TKD mutation is an FLT 835 mutation, such as D835H, D835V, or D835Y. In particular embodiments, the TKD mutation comprises an FLT3 691 mutation, such as F691L.

In some aspects of the second embodiment, the FLT3 mutation is an ITD mutation and a TKD mutation, such as ITD-835H, ITD-835V, ITD-D835Y, or ITD-F691L.

Aspects of the second embodiment may include administering pacritinib, or a pharmaceutically acceptable salt, prodrug, or N-oxide thereof. In particular embodiments, the pharmaceutically acceptable salt is a citrate salt or a maleate salt.

Certain aspects of the second embodiment include administering an effective amount of pacritinib, or a pharmaceutically acceptable salt, prodrug, or N-oxide thereof. Pacritinib may be effective over a wide dosage range. In certain embodiments, the effective amount ranges from 0.01 to 2000 mg, from 1 to 1000 mg per day, from 50 to 500 mg per day, or from 200 to 400 mg per day. In particular embodiments, the effective amount ranges from between about 50 mg per day and about 500 mg per day, or is about 200 mg per day. In various embodiments, the effective amount is 50, 100, 150, 200, 250, 300, 350, 400, 450, or 500 mg per day.

Some aspects of the second embodiment provide methods including administering one or more further therapeutic agents, such as a chemotherapeutic agents, therapeutic antibodies, and radiation treatment, to provide a synergistic or additive therapeutic effect.

Certain aspects of the second embodiment further include administering an effective amount of one or more chemotherapeutic agents. Many chemotherapeutic agents are presently known in the art and can be used in combination with pacritinib, or a pharmaceutically acceptable salt, prodrug, or N-oxide thereof. In some embodiments, the chemotherapeutic agent is selected from the group consisting of mitotic inhibitors, alkylating agents, anti-metabolites (e.g., nucleoside analogs), intercalating agents, growth factor inhibitors, cell cycle inhibitors, enzymes, topoisomeRASe inhibitors, biological response modifiers, anti-hormones, angiogenesis inhibitors, hypomethylating agents, and anti-androgens.

Non-limiting examples of chemotherapeutic agents that can be used in some aspects of the second embodiment in combination with pacritinib, or a pharmaceutically acceptable salt, prodrug, or N-oxide thereof are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above.

In some aspects of the second embodiment, the one or more further therapeutic agents comprises a nucleoside analog or an intercalating agent. In other embodiments, the one or more further therapeutic agents comprise a nucleoside analog and an intercalating agent. In embodiments, the nucleoside analog may be: a purine analog such as fludarabine, 6-mercaptopurine, thiamiprine, thioguanine; or a pyrimidine analog, such as ancitabine, azacitidine, 6-azauridine, carmofur, cytarabine (e.g., Ara-C, dideoxyuridine, doxifluridine, enocitabine, or floxuridine. In particular embodiments, the nucleoside analog comprises cytarabine.

In aspects of the second embodiment, the intercalating agent is doxorubicin, daunorubicin (also known as daunomycin), or dactinomycin. In particular embodiments, the intercalating agent is daunorubicin.

In some aspects of the second embodiment, the one or more further therapeutic agents comprises a hypomethylating agent. In particular embodiments, the hypomethylating agent comprises decitabine (also known as 5-aza-2′-deoxycytidine) or azacytidine. Decitabine may be given, for example, to a patient with relapsed or refractory AML, and/or to patients who are ineligible for a more intensive therapy regimen. Decitabine may be administered, for example, at 20 mg/m², four times every twenty four hours, on days 1-10 of a 28 day treatment cycle.

When used in combination with one or more further therapeutic agents in aspects of the second embodiment, pacritinib, or a pharmaceutically acceptable salt, prodrug, or N-oxide thereof is administered with the second agent simultaneously or separately. This administration in combination can include simultaneous administration of the two agents in the same dosage form, simultaneous administration in separate dosage forms, and separate administration. In some embodiments, pacritinib, or a pharmaceutically acceptable salt, prodrug, or N-oxide thereof, and any of the agents described above (e.g., cytarabine, daunorubicin, and/or decitabine) can be simultaneously administered, wherein both the agents are present in separate formulations. In another alternative, pacritinib, or a pharmaceutically acceptable salt, prodrug, or N-oxide thereof can be administered just followed by any of the agents described above, or vice versa. In some embodiments of the separate administration protocol pacritinib, or a pharmaceutically acceptable salt, prodrug, or N-oxide thereof, and any of the agents described above are administered a few minutes apart, or a few hours apart, or a few days apart.

Aspects of the second embodiment described herein may include treating acute myeloid leukemia (AML). In particular embodiments, the AML may be newly diagnosed AML, or relapsed or refractory AML (i.e., AML that does not undergo remission following an initial treatment). In certain specific embodiments, the acute myeloid leukemia is relapsed or refractory acute myeloid leukemia.

A third embodiment of the present invention provides a method of selecting a treatment regimen for a subject in need of treatment for a hematologic cancer, the method comprising: (i) receiving a genetic profile for the subject, the genetic profile comprising an FLT3 mutation; and (ii) selecting a treatment regimen based on the genetic profile, the treatment regimen comprising a therapeutic agent having inhibitory activity against Janus kinase 2 (JAK2) and fms-like tyrosine kinase 3 (FLT3).

Such genetic profiles may be produced in any suitable manner (e.g., microarrays, reverse transcription polymerase chain reaction (RT-PCR), etc.).

In some aspects of the third embodiment the FLT3 mutation is an internal tandem duplication (ITD) mutation. The ITD mutation may be any tandem duplication within the juxtamembrane domain of FLT3.

In certain aspects of the third embodiment, the activating FLT3 mutation is a tyrosine kinase domain (TKD) mutation. In particular embodiments, the TKD mutation is an FLT 835 mutation, such as D835H, D835V, or D835Y. In particular embodiments, the TKD mutation comprises an FLT3 691 mutation, such as F691L.

In certain aspects of the third embodiment, the FLT3 mutation comprises an ITD mutation and a TKD mutation. In particular embodiments, the FLT3 mutation comprises ITD-835H, ITD-835V, ITD-D835Y, or ITD-F691L.

The treatment regimen in aspects of the third embodiment may include a therapeutic agent having inhibitory activity against JAK2 and FLT3. In certain embodiments, the therapeutic agent is pacritinib, or a pharmaceutically acceptable salt, prodrug, or N-oxide thereof. In particular embodiments, the pharmaceutically acceptable salt is a citrate salt or a maleate salt.

In certain aspects of the third embodiment, the treatment regimen includes administering an effective amount of a therapeutic agent, such as pacritinib. Pacritinib may be effective over a wide dosage range. In certain embodiments, the effective amount ranges from 0.01 to 2000 mg, from 1 to 1000 mg per day, from 50 to 500 mg per day, or from 200 to 400 mg per day. In particular embodiments, the effective amount ranges from between about 50 mg per day and about 500 mg per day, or is about 200 mg per day. In various embodiments, the effective amount is 50, 100, 150, 200, 250, 300, 350, 400, 450, or 500 mg per day.

In some aspects of the third embodiment, the treatment regimen includes a therapeutic agent having inhibitory activity against JAK2 and FLT3 and one or more further therapeutic agents, such as a chemotherapeutic agents, therapeutic antibodies, and radiation treatment, to provide a synergistic or additive therapeutic effect.

In certain aspects of the third embodiment, the treatment regimen includes administering a therapeutic agent having inhibitory activity against JAK2 and FLT3 and one or more chemotherapeutic agents. Many chemotherapeutic agents are presently known in the art and can be used in combination with a therapeutic agent having inhibitory activity against JAK2 and FLT3. In some embodiments, the chemotherapeutic agent is selected from the group consisting of mitotic inhibitors, alkylating agents, anti-metabolites (e.g., nucleoside analogs), intercalating agents, growth factor inhibitors, cell cycle inhibitors, enzymes, topoisomeRASe inhibitors, biological response modifiers, anti-hormones, angiogenesis inhibitors, hypomethylating agents, and anti-androgens.

Non-limiting examples of therapeutic agents that can be used in aspects of the third embodiment in combination with a therapeutic agent having inhibitory activity against JAK2 and FLT3 are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine;

arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above.

In some aspects of the third embodiment, the one or more further therapeutic agents comprises a nucleoside analog or an intercalating agent. In embodiments, the nucleoside analog may be: a purine analog such as fludarabine, 6-mercaptopurine, thiamiprine, thioguanine; or a pyrimidine analog, such as ancitabine, azacitidine, 6-azauridine, carmofur, cytarabine (e.g., Ara-C, dideoxyuridine, doxifluridine, enocitabine, or floxuridine. In particular embodiments, the nucleoside analog comprises cytarabine.

In aspects of the third embodiment, the intercalating agent is doxorubicin, daunorubicin (also known as daunomycin), or dactinomycin. In particular embodiments, the intercalating agent is daunorubicin.

In other aspects of the third embodiment, the one or more further therapeutic agents comprise a nucleoside analog and an intercalating agent. For example, treatment with cytarabine and daunorubicin is a common chemotherapy regiment for AML. In particular embodiments, the cytarabine and daunorubicin are administered as a “7+3” (or “3+7”) regimen. For a “7+3” regimen, daunorubicin may be replaced with doxorubicin, idarubicin, or mitoxantrone. For a “7+3” regimen, daunorubicin may be administered, for example, at 60 mg/m², four times every twenty four hours, on days 1-3 of a 21 day treatment cycle, and cytarabine may be administered, for example, at 100 g/m² four times every twenty hours, on days 1-7 of a 21 day treatment cycle.

In some aspects of the third embodiment, the one or more further therapeutic agents comprises a further therapeutic agent having activity against FLT3-mutated cancer cells. In particular embodiments, the further therapeutic agent comprises midostaurin.

In some aspects of the third embodiment, the one or more further therapeutic agents comprises a hypomethylating agent. In particular embodiments, the hypomethylating agent comprises decitabine (also known as 5-aza-2′-deoxycytidine) or azacytidine. Decitabine may be given, for example, to a patient with relapsed or refractory AML, and/or to patients who are ineligible for a more intensive therapy regimen. Decitabine may be administered, for example, at 20 mg/m², four times every twenty four hours, on days 1-10 of a 28 day treatment cycle.

When used in combination with one or more further therapeutic agents, a therapeutic agent having inhibitory activity against JAK2 and FLT3 is administered with the second agent simultaneously or separately. This administration in combination can include simultaneous administration of the two agents in the same dosage form, simultaneous administration in separate dosage forms, and separate administration. In some embodiments, a therapeutic agent having inhibitory activity against JAK2 and FLT3, and any of the agents described above (e.g., cytarabine, daunorubicin, and/or decitabine) can be simultaneously administered, wherein both the agents are present in separate formulations. In another alternative, a therapeutic agent having inhibitory activity against JAK2 and FLT3, can be administered just followed by any of the agents described above, or vice versa. In some embodiments of the separate administration protocol a therapeutic agent having inhibitory activity against JAK2 and FLT3, and any of the agents described above are administered a few minutes apart, or a few hours apart, or a few days apart.

Aspects of the third embodiment described herein may include treating a hematologic cancer, such as multiple myeloma, myelodysplastic syndrome (MDS), acute myeloid leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma. In some specific embodiments, the hematologic cancer is CLL, SLL, or both. In some specific embodiments, the hematologic cancer is acute myeloid leukemia (AML). In particular embodiments, the AML may be newly diagnosed AML, or relapsed or refractory AML (i.e., AML that does not undergo remission following an initial treatment). In certain specific embodiments, the acute myeloid leukemia is relapsed or refractory acute myeloid leukemia.

A fourth embodiment of the present invention provides a method of inhibiting FLT3 activity in a cell with an FLT3 mutation, the method comprising contacting the cell with an effective amount of pacritinib, or a pharmaceutically acceptable salt, prodrug, or N-oxide thereof.

Such genetic profiles may be produced in any suitable manner (e.g., microarrays, reverse transcription polymerase chain reaction (RT-PCR), etc.).

In some aspects of the fourth embodiment, the FLT3 mutation comprises an internal tandem duplication (ITD) mutation. The ITD mutation may be any tandem duplication within the juxtamembrane domain of FLT3. In some embodiments, the FLT3 mutation comprises a tyrosine kinase domain (TKD) mutation. In some specific embodiments, the FLT3 mutation comprises an ITD mutation and a TKD mutation. In particular embodiments, the FLT3 mutation is ITD, D835H, D835V, D835Y, ITD-835H, ITD-835V, ITD-D835Y, or ITD-F691L.

Contacting the cell with an effective amount of pacritinib, or a pharmaceutically acceptable salt, prodrug, or N-oxide thereof, may be performed in vitro or in vivo.

In some aspects of the fourth embodiment, the effective amount of pacritinib for inhibiting FLT3 activity in a cell with an FLT3 mutation is between about 10 nM and 1,000 nM, between about 50 nM and about 800 nM, or between about 100 nM and about 500 nM.

In aspects of the fourth embodiment, the pacritinib may inhibit FLT3 activity in a cell with an FLT3 mutation with an IC₅₀ value of less than 500 nM, less than 400 nM, less than 300 nM, less than 200 nM, less than 100 nM, or less than 50 nM.

In aspects of the fourth embodiment, the pacritinib may bind to the mutated FLT3 with a binding affinity (expressed as Kd), of less than 100 nM, less than 50 nM, or less than 20 nM.

In some aspects of the fourth embodiment, the cell is a hematopoietic cell. In particular embodiments, the cell is an acute myeloid leukemia cell.

In some aspects of the fourth embodiment, the inhibiting FLT3 activity causes an anti-cancer effect. An agent that causes an anti-cancer effect can, for example, selectively cause reduced proliferation or cellular toxicity to cancer cells but not healthy, non-cancerous cells. An anti-cancer effect may be, for example, a statistically significant reduction in the number of cancer cells (e.g., at least a 20% reduction, at least a 25% reduction, at least a 30% reduction, at least a 35% reduction, at least a 40% reduction, at least a 45% reduction, or at least a 50% reduction), but a smaller reduction in (or no reduction in) non-cancer cells, following contacting the cells with the effective amount of the compound. The non-cancer cells may be a relevant type of control cells, such as non-cancerous myeloid cells.

The examples provided below further illustrate and exemplify methods of using a compound of Formula (I), such as pacritinib, to treat a subject or cells having a mutation in FLT3. It is to be understood that the scope of the present disclosure is not limited in any way by the scope of the following examples.

### EXAMPLES

### Example 1

### Study of Pacritinib and Chemotherapy in Patients with Acute Myeloid Leukemia and FLT3 Mutations

This is a study evaluating pacritinib and chemotherapy in adult AML patients with FLT3 mutations (ITD or TKD). Patient samples will be screened for the presence of FLT3 mutations (ITD and/or TKD). Adult AML patients having an FLT3 mutation (ITD and/or TKD) will place placed in two different cohorts, as follows:

Cohort A: Fit untreated AML patients (>18 years, core-binding factor negative) with FLT3 mutations will receive an intensive therapy combination using pacritinib with cytarabine and daunorubicin (“7+3”).

Cohort B: Unfit patients>60 years who are not considered candidates for intensive chemotherapy by physician opinion, with untreated AML with FLT3 mutations or any AML patient with FLT3 mutation and relapsed or refractory disease, will receive a lower-intensity therapy combination using pacritinib with decitabine.

Treatment Plan

COHORT A: Pacritinib with cytarabine and daunorubicin (7+3) for untreated AML patients fit for intensive therapy and ≥18 years

COHORT B: Pacritinib with decitabine for untreated AML patients>60 years unfit for intensive therapy or for AML patients with relapsed or refractory disease

Patients age≥18 with untreated AML and the presence of FLT3 mutation may enroll to Cohort A only. Untreated AML patients>60 years who are not candidates for intensive chemotherapy and patients with relapsed or refractory FLT3-mutated AML may enroll to Cohort B. Patients with secondary AML or therapy related disease (t-AML) are eligible.

Drug Administration. Pacritinib will be prepared for oral administration as size #0 hard gelatin capsules with gray bodies and red caps. Capsules will contain 100 mg pacritinib (free base) and the following inactive ingredients: microcrystalline cellulose NF, polyethylene glycol 8000 (PEG 8000) NF, and magnesium stearate NF. The capsule gelatin will be bovine-derived.

Treatment will be administered on an inpatient basis for Cohort A and inpatient or outpatient basis for Cohort B depending on the treating physician's decision.

COHORT A: Pacritinib with cytarabine and daunorubicin (7+3) for untreated AML, patients fit for intensive therapy and ≥18 years.

TABLE 1
Induction(s) for cohort A

| | Pacritinib | Cytarabine | Daunorubicin |
|---|---|---|---|
| Dose | (D 1-4 and D 8-21) | (D 5-11) | (D 5-7) |
| Level | (mg/day PO) | (g/m² q 24 h) | (mg/m² q 24 h) |
| 1 | 200 (100 mg BID) | 100 | 60 |
| 2 | 300 (200 mg in AM, | 100 | 60 |
| | 100 mg in PM) | | |
| 3 | 400 (200 mg BID) | 100 | 60 |

Patients in cohort A will receive: pacritinib in an oral dose of either 200 mg per day, 300 mg per day, or 400 mg per day, on days 1 through 4 and days 8 through 21 of the 28-day induction cycle; intravenous cytarabine at a dose of 100 g/m² every 24 hours on days 5 through 11 of the 28-day induction cycle; and intravenous daunorubicin at a dose of 60 mg/m² every 24 hours on days five through 7 of the 28-day induction cycle.

On the first day of pacritinib treatment, the second (e.g. evening) dose of pacritinib will be held to perform a 24-hour plasma PK study. Twice daily pacritinib administration will begin on day 2. A bone marrow aspiration and biopsy will be performed at count recovery (ANC>1000/μL, platelets>100,000/μL) or by day 35 (whichever is the earlier time point). Patients with persistent disease may receive a second induction cycle with the same doses as above; however, patients will receive cytarabine on days 1-7, daunorubicin on days 1-3 and pacritinib (200 mg/day) on days 4-25. Patients achieving complete remission (CR) or who fail to achieve CR after two inductions will be removed from study to receive consolidation/salvage chemotherapy as indicated.

Cohort A monitoring and assessment of disease response: Bone marrow (BM) aspirations and biopsies are required before treatment and at count recovery (ANC>1,000/μL, and platelet count≥100,000/μL) or by day 35 (whichever is the earlier time point). Patients who achieve CR/CRi, if eligible, will be evaluated for allogeneic hematopoietic stem cell transplant or additional courses of chemotherapy (high-dose cytarabine) at the discretion of the treating physician.

COHORT B: Pacritinib with decitabine for untreated AML patients>60 years unfit for intensive therapy or for AML patients with relapsed or refractory disease.

TABLE 2
First Induction for cohort B.

| | Pacritinib | Decitabine |
|---|---|---|
| Dose | (D 1-21) | (D 5-14) |
| Level | (mg/day PO) | (mg/m² q 24 h) |
| 1 | 200 (100 mg BID) | 20 |
| 2 | 300 (200 mg in AM | 20 |
| | and 100 mg in PM) | |
| 3 | 400 (200 mg BID) | 20 |

Patients in cohort B will receive pacritinib at a dose of either 200 mg per day, 300 mg per day, or 400 mg per day, on days 1 through 21 of the first 28-day induction cycle; and a continuous intravenous infusion of decitabine at a dose of 20 mg/m² every 24 hours on days 5 through 14 of the first 28-day induction cycle.

Induction Cycles 2-4 (if necessary): Patients may receive up to four 28-day cycles. The doses for induction cycles 2-4 will be the same as the first induction, but however both drugs will begin together on day 1 of cycles 2-4 (there will be no 4-day lead-in phase with pacritinib alone).

Maintenance (<5% blasts detected in marrow): Patients achieving CR will proceed with transplant evaluation (if appropriate). Transplant-ineligible patients will receive maintenance courses of decitabine 20 mg/m2 IV over 1 hour daily on days 1-5 and pacritinib on days 1-21. Cycles will be repeated every 28 days until disease progression or until development of unacceptable toxicities warranting discontinuation of further therapy. Subsequent cycles of decitabine may be dose reduced to 4 or 3 days of decitabine/cycle based on myelosuppression. Pacritinib may be continued for up to 2 years in the patients who remain in remission.

Cohort B monitoring and assessment of disease response: A cycle will be defined as 4 weeks (28 days) for induction and maintenance. A repeat bone marrow (BM) aspiration and biopsy is required if treatment is delayed more than 2 weeks, unless the patient has circulating blasts in the peripheral blood. Bone marrow (BM) aspirations and biopsies are required before treatment and at count recovery (ANC>1,000/μL, and platelet count≥100,000/μL) or by day 32 (induction cycle 1 only). For induction cycles 2 through 4, patients with circulating blasts in peripheral blood at the end of each cycle do not require BM aspirates and biopsies; however, patients with pancytopenia without blasts should be evaluated for response. During maintenance, BM examination is indicated only for patients with evidence of myelosuppression or disease recurrence at the discretion of the treating physician.

Treatment will consist of 1-2 cycles of induction therapy (Cohort A) or up to four induction cycles (Cohort B). Patients in Cohort B will continue maintenance therapy after remission is achieved until disease response is lost.

During and following the treatment cycles, patients will be assessed for disease response.

Disease Response Assessment Criteria:

### Morphologic Complete Remission (Morphologic CR)

Morphologic CR requires all of the following:

<5% blasts in bone marrow aspirate.

Neutrophils>1,000/μL.

Platelets>100,000/μL.

No extramedullary disease.

No blasts with Auer rods detected.

No circulating blasts (rare may be permitted)/No evidence of pre-treatment blast phenotype by flow cytometry (i.e. CD34, CD7 co-expression)

### Morphologic Leukemia-Free State (MLFS)

MLFS requires all of the following:

<5% blasts in a bone marrow aspirate sample with marrow spicules and a count of at least 200 nucleated cells

No blasts with persistence of Auer rods

No extramedullary disease

### Cytogenetic Complete Remission (CRc)

CRc requires all of the following:

<5% blasts in bone marrow aspirate.

Neutrophils>1,000/μL.

Platelets>100,000/μL.

No extramedullary disease.

No blasts with Auer rods detected.

No circulating blasts (rare may be permitted)/No evidence of pre-treatment blast phenotype by flow cytometry (i.e. CD34, CD7 co-expression)

Reversion to a normal karyotype.

Morphologic CR with Incomplete Blood Count Recovery (CRi)

CRi requires all of the following:

<5% blasts in bone marrow aspirate.

Neutrophils<1,000/μL.

Platelets<100,000/μL.

No extramedullary disease.

No blasts with Auer rods detected.

No circulating blasts (rare may be permitted)/No evidence of pre-treatment blast phenotype by flow cytometry (i.e. CD34, CD7 co-expression)

### Partial Remission (PR)

PR requires all of the following:

Meets all criteria for CR except for BM blasts

Must have greater than 50% decrease in blasts in bone marrow aspirate to a range of 5-25%.

Neutrophils>1,000/μL.

Platelets>100,000/μL.

No extramedullary disease.

### Progressive Disease (PD)

PR requires all of the following:

Presence of >50% increase in bone marrow blasts to a level of at least 50% and/or a doubling of the percentage of peripheral blood blasts to a level of at least 50%.

Stable Disease (SD): Not fulfilling criteria for CR, CRi, PR, or disease progression

### Example 2

### Preclinical Activity of Pacritinib on FLT3-Mutated Cells

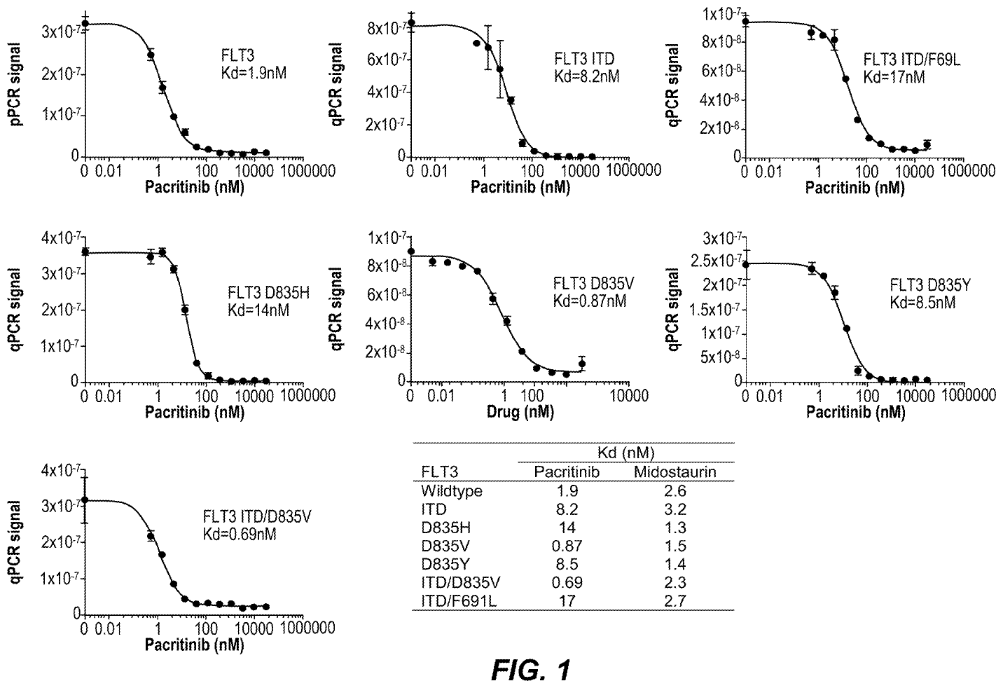

For preclinical studies, drug binding affinities to FLT3 wildtype, ITD, and TKD mutant kinases at residues F691 and D835 were evaluated using the KdELECT assay. Drug and DMSO control were tested in the concentration range of 0.003-30,000 nM. The binding constants (Kds) were calculated with a standard dose-response curve using the Hill equation and a non-linear square fit with the Levenberg-Marquardt altorigthm. FIG. 1 shows the binding affinities of pacritinib to various FLT3 variants. Binding affinities to FLT3 wildtype and all FLT3 mutants expressed as Kd were: FLT3 WT (1.9 nM), ITD (8.2 nM), D835H (14 nM), D835V (0.87 nM), D835Y (8.5 nM), ITD/D835Y (0.69 nM), and ITD/F691L (17 nM).

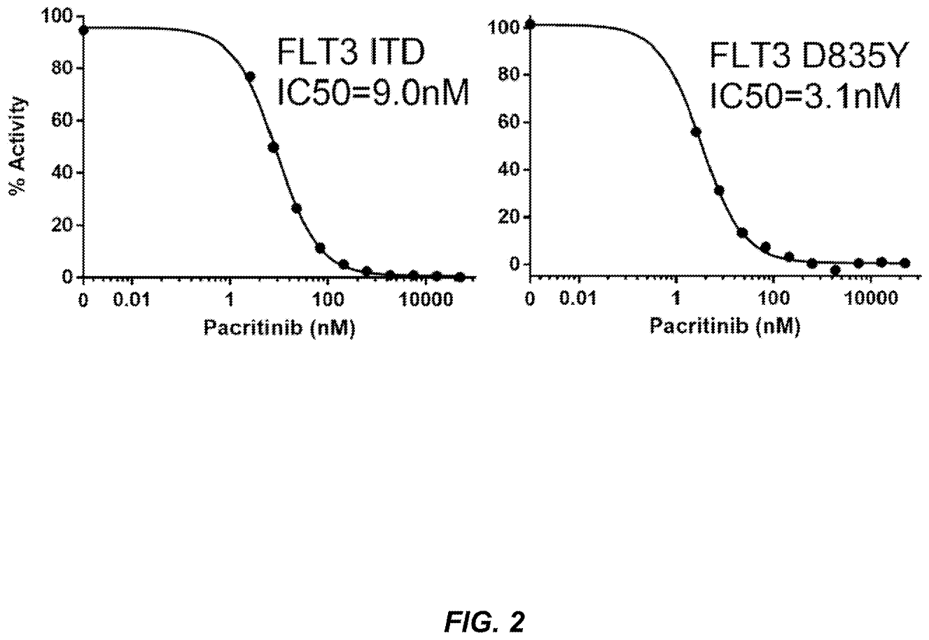

Kinase inhibition activity against ITD and D835Y mutant FLT3 was evaluated using the HotSpot Kinase assay. Drug and control was tested in the concentration range of 2.5-50,000 nM. Enzymatic activity was measured relative to DMSO control and a standard dose response with the Hill equation was used to calculate IC50. In the kinase assays, pacritinib inhibited FLT3 ITD and D835Y with IC₅₀ values of 9 nM and 3.1 nM, respectively (see FIG. 2).

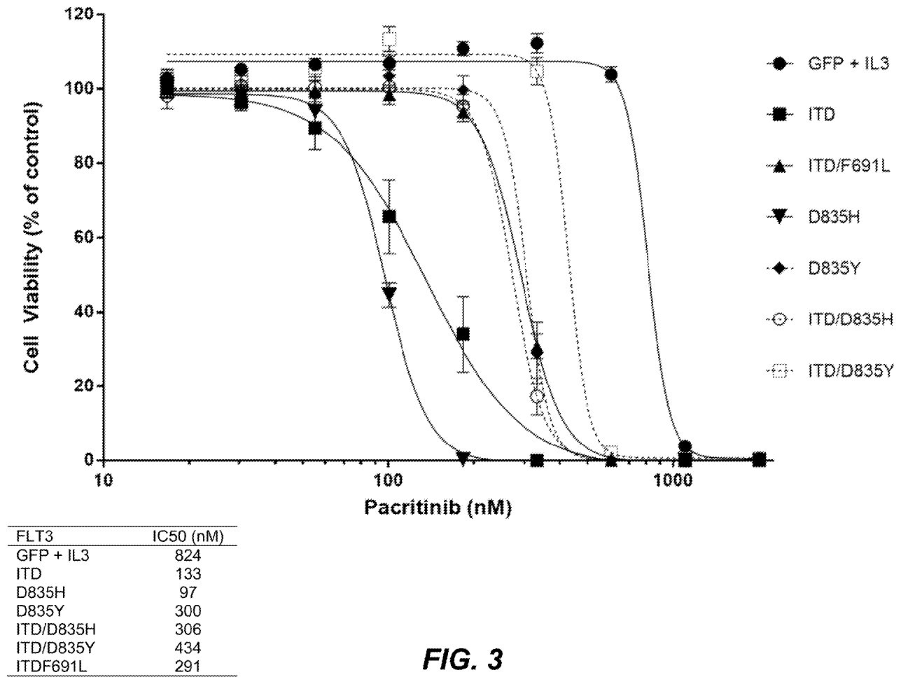

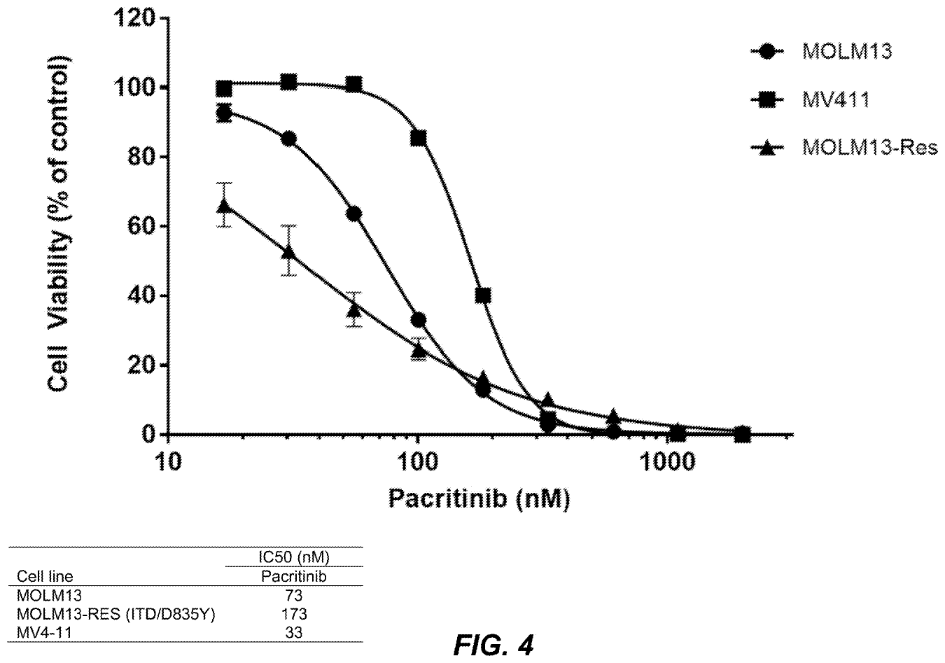

The effect of pacritinib on growth inhibition was assessed in Ba/F3, murine interleukin-3 dependent pro-B cells, transfected with FLT3-ITD and TKD mutants, FLT3-ITD+ AML cell lines (MOLM13 and MV4-11), and FLT3-ITD/D835Y AML cells (MOLM13-Res) after 72 hours of drug treatment. MOLM13 has a 21 base pair internal tandem duplication in the juxtamembrane domain of FLT3, and MV4-11 has a 30 base pair tandem duplication in the juxtamembrane domain of FLT3 (Carson et al., Blood 2013 122:1382). The activity of pacritinib and in Ba/F3 cells transfected with vector control (VC) or different FLT3 mutants and pacritinib and midostaurin in three FLT3-ITD+ AML cell lines was evaluated using MTT assay (Roche Diagnostics, Mannheim, Germany) as described previously in Zimmerman, E.I., et al. Blood 122, 3607-3615 (2013); and Jarusiewicz, J. A., et al. ACS Omega 2, 1985-2009 (2017). Results were measured as a mean percentage of DMSO-treated control cells at each concentration (performed in 3-6 replicates). As shown in FIG. 3, pacritinib inhibited the viability of Ba/F3 transfected with GFP (mean IC₅₀ of 2 independent experiments, 824 nM), and activity was more potent against all cells expressing different FLT3 mutants: FLT3-ITD (133 nM), D835H (97 nM), D835Y (300 nM), ITD/D835H (306 nM), ITD/D835Y (434 nM), and ITD/F691L (291 nM). As shown in FIG. 4, in FLT3-ITD⁺ AML cell lines, pacritinib had activity with mean IC₅₀ values of 73 nM, 173 nM, and 33 nM in MOLM13, MOLM13-Res, and MV4-11 cells, respectively.

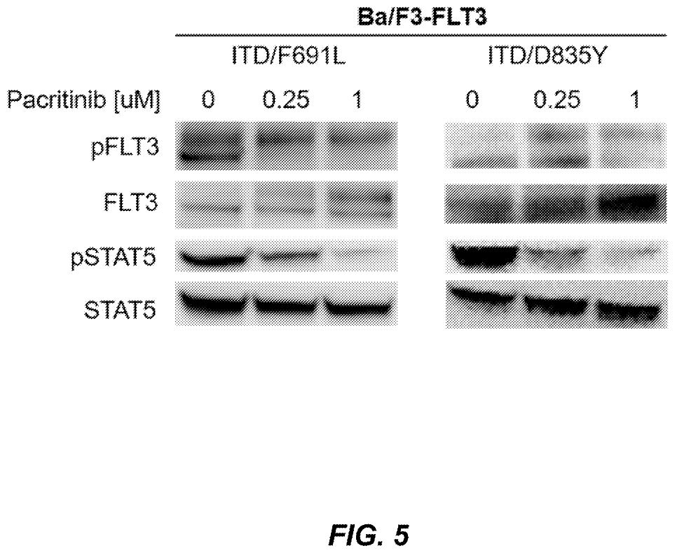

Next, inhibition of FLT3 signaling in Ba/F3 cells was determined by Western blot analysis. Cells were treated with pacritinib or DMSO for 4 hours and lysed in RIPA buffer supplemented with protease and phosphatase inhibitors. Immunoprecipitation was carried out overnight with anti-FLT3 antibodies (Santa Cruz Biotechnology, Dallas, Tex.). Dynabeads (ThermoFisher, Waltham, Mass.) were added the next day and incubated for 4 hours. Samples were then prepared according to manufacturer's instructions. Eluted lysates or total cell lysates were separated by SDS-polyacrylamide gel electrophoresis and transferred to PVDF membranes followed by Western blot analysis using the indicated primary antibodies: FLT3, phospho-FLT3, STAT5, and phospho-STAT5 (Cell Signaling Technology, Danvers, Mass.). As seen in FIG. 5, western blot analysis of lysates from pacritinib-treated Ba/F3 cells expressing ITD/D835Y or ITD/F691L mutants showed inhibition of phospho-FLT3 and phospho-STAT5 at 0.25-1 uM.

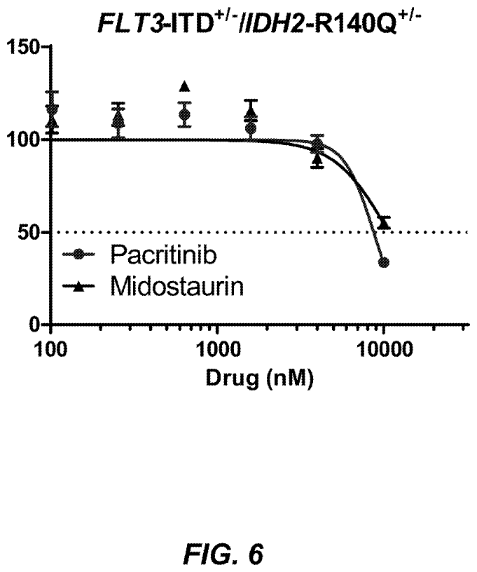

Mutations in IDH2 commonly co-occur with FLT3 mutations in AML. Thus, viability of cells harboring IDH2 and an FLT3 mutation was tested. In murine primary leukemia cells with double knockin of FLT3-ITD+/−/IDH2-R140Q+/−, mutations that co-occur in AML, pacritinib inhibited cell viability with an IC50 value of 8.7 μM, while midostaurin had an IC50 value of 10.7 μM (FIG. 6).

Previous reports have shown pacritinib to be active against de novo FLT3-ITD+ AML models in cell lines, primary cells, and a cell line xenograft⁸. Therefore, the anti-leukemic activity of pacritinib was assessed in various models of drug resistant of FLT3-ITD+ AML. In binding and kinase assays, pacritinib retained activity against FLT3 TKD mutants compared to FLT3-ITD. In Ba/F3 cells expressing FLT3 mutant proteins, sensitivity of pacritinib remained similar between the ITD or TKD mutants and dual ITD/TKD mutants, and inhibited phospho-FLT3 and its downstream mediator, phospho-STAT5. Furthermore, in MOLM13-Res (ITD/D835Y+) AML cells, the pacritinib IC50 value did not show a large shift compared to that in parental MOLM13 (ITD+) cells. This is in contrast to the type II FLT3 inhibitors sorafenib and quizartinib, which become highly resistant in AML cells with TKD mutants. Given the frequent co-occurrence of additional mutations with FLT3-ITD in AML patients, it is worth highlighting that pacritinib had similar activity to midostaurin against murine primary leukemia cells harboring FL T3-ITD along with an IDH2-R140Q mutation. Collectively, this in vitro assessment of pacritinib shows that pacritinib has type I inhibitor properties with activity against FLT3 inhibitor resistant forms of FLT3-ITD+ AML. Additionally, these results show that pacritinib can inhibit a variety of FLT3 mutants, such as various TKD mutants.

### Example 3

### Clinical Study of Pacritinib and Chemotherapy in FLT3-ITD+ AML

Phase I study summary. In the phase I study, the dose of pacritinib (100 mg BID or 200 mg BID) was escalated following a 3+3 design and was administered with cytarabine and daunorubicin in a 7+3 regimen (Cohort A) or with decitabine (Cohort B) on the following schedules: pacritinib days 1-21, cytarabine days 5-11, daunorubicin days 5-7, and decitabine days 5-14. Five patients have been enrolled to Cohort A and eight patients have been enrolled to Cohort B. Plasma samples for pacritinib pharmacokinetic studies were obtained on days 1 and 21 at pretreatment and after pacritinib administration at 1, 2, 3, 5, 24 hours; a pretreatment sample was obtained on day 5. Pacritinib was quantitated in plasma using a validated LC/MS-MS bioanalytical assay. Ex vivo sensitivity of pacritinib (N=4) and analysis of TKD mutations in FLT3 exons 17 and 20 by deep amplicon sequencing (N=13) was performed in pretreatment bone marrow primary blast samples.

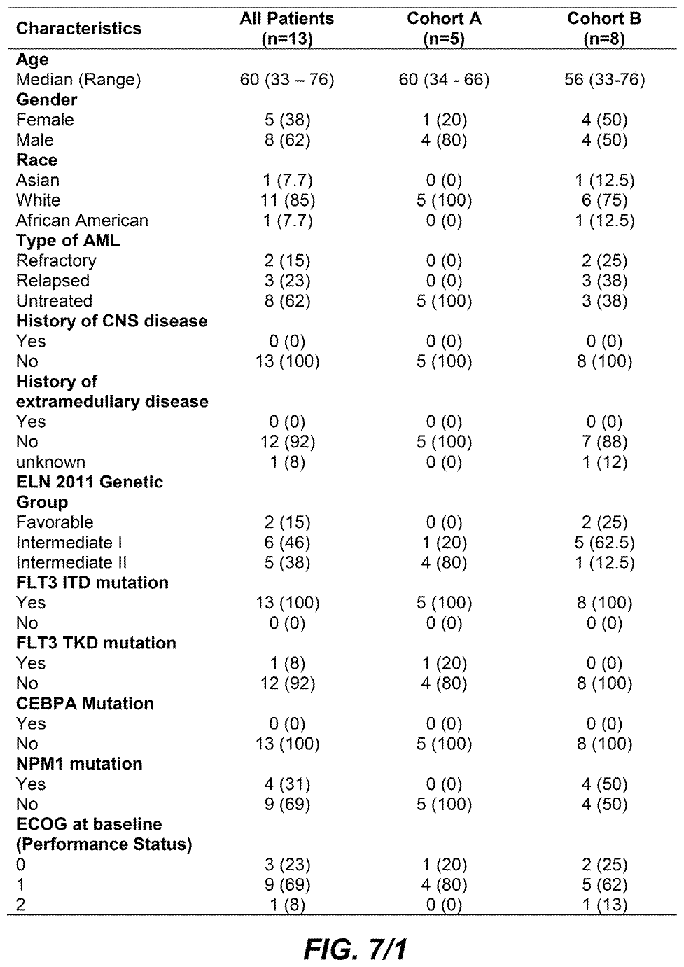

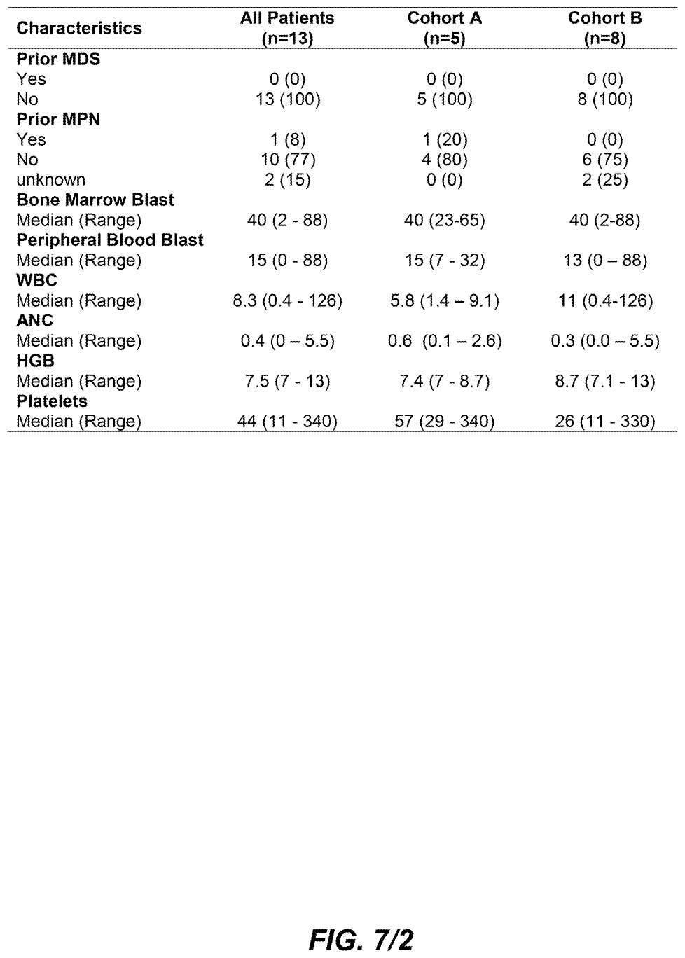

Patient population. Patients>18 years old with a histologically confirmed diagnosis of untreated AML (excluding acute promyelocytic leukemia), or those with relapsed or refractory AML (cohort B only), Eastern Cooperative Oncology Group (ECOG) status<2, with the presence of FLT3 mutations were eligible for enrollment. Patients were required to have adequate organ function defined as total bilirubin of 2.0 mg/dL or less unless due to Gilbert's disease, aspartate aminotransferase and alanine aminotransferase of 2.5× the upper limit of normal or less, creatinine clearance of 50 mL/min or greater by Cockcroft-Gault, New York Heart Association (NYHA) congestive heart failure (CHF) class II or better, and left ventricular ejection fraction of 50% or greater. Patient life expectancy was required to be greater than 6 months when present with co-morbid illnesses. Patient demographics and baseline characteristics are shown in FIG. 7.

Exclusion criteria included patients who had core-binding factor AML with (inv(16), t(8;21)); patients with uncontrolled intercurrent illness including but not limited to symptomatic CHF, unstable angina, myocardial infarction within 6 months, severe uncontrolled ventricular arrhythmias or electrocardiographic evidence of acute ischemia or active conduction system abnormalities; pregnant or breastfeeding women; baseline QTc greater than 450 ms or patients taking medications that prolong QTc interval; patients who received potent cytochrome P450 3A4 (CYP3A4) inhibitor 1 week prior to treatment; and use of concomitant potent CYP3A4 inducers. Additional exclusion criteria included: Patients receiving any other investigational agents or that have received other investigational agents within 14 days of enrollment; patients with significantly decreased or obstructed gastrointestinal tract; patients with serious medical psychiatric illness that could interfere with participation; patients who had chemotherapy or radiotherapy or major surgery within 2 weeks prior to entering the study; patients with active central nervous system malignancy; patients with a history of platelet alloimmunization; patients with other malignancy within the last 3 years, other than curatively treated basal cell or squamous cell skin cancer, carcinoma in situ of the cervix, organ confined or treated nonmetastatic prostate cancer with negative prostate-specific antigen, in situ breast carcinoma after complete surgical resection, or superficial transitional cell bladder carcinoma; and patients who are not able to swallow capsules or tablets; known active HIV or hepatitis A, B, or C virus infection.

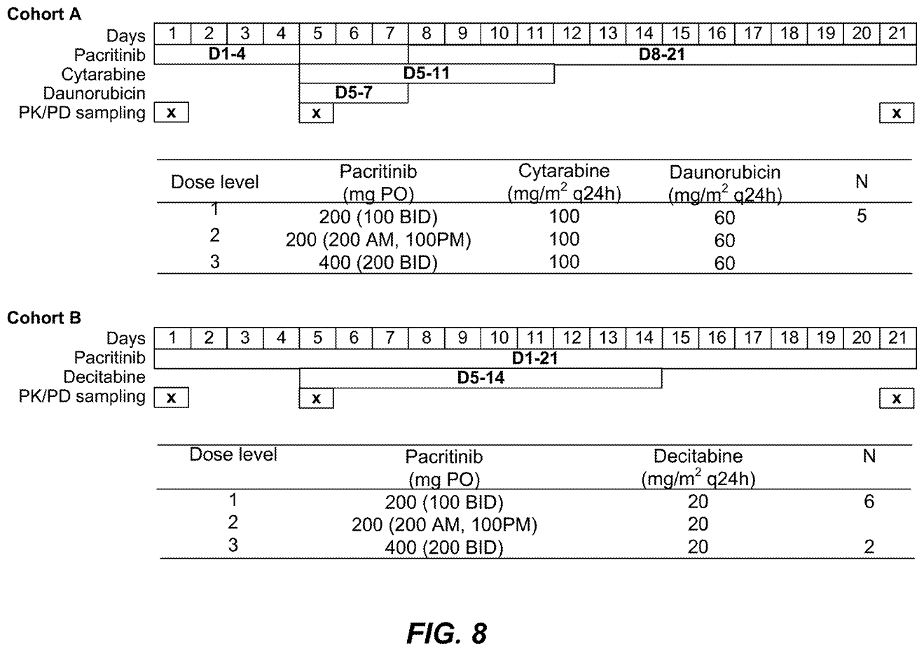

Treatment plan. Patients were treated in parallel in one of two cohorts (FIG. 8). Fit patients who were eligible for intensive chemotherapy were assigned to cohort A and received pacritinib on days 1-4 and days 8-21, cytarabine 100 mg/m² days 5-11, and daunorubicin 60 mg/m² days 5-7. Patients>60 years, who were considered unfit for intensive therapy, and those with relapsed/refractory disease, were assigned to cohort B receiving pacritinib days 1-21 and decitabine 20 mg/m² days 5-14. Initially, patients were treated with pacritinib 200 mg twice daily (dose level 1). However, due to safety concerns, pacritinib was temporarily placed on a full clinical hold by the Food and Drug Administration. At the time of the clinical hold, two patients had been enrolled in Cohort B. Once the hold was lifted, the protocol was amended to change the study design to a standard 3+3 dose-escalation design. Following the amendment, patients treated on dose level 1 received 200 mg of pacritinib per day (100 mg by mouth twice a day). Patients on dose level 2 received 300 mg per day (200 mg in the morning and 100 mg in the evening) and patients on dose level 3 received 400 mg of pacritinib per day (200 mg twice a day). Treatment consisted of 1-2 cycles of induction therapy (cohort A) or up to 4 induction cycles (cohort B). Patients in cohort A who achieved complete remission (CR) were evaluated for hematopoietic stem cell transplant or additional courses of chemotherapy at the discretion of the clinician. Patients in cohort B who achieved CR were able to proceed with transplant, if eligible, or 5-day maintenance courses of decitabine and pacritinib. Patients received full supportive care including transfusions of blood and blood products, antibiotics, and antiemetics when appropriate.

Safety Assessments. This study utilized the National Cancer Institute Common Terminology Criteria for Adverse Events version 4.03 to characterize toxicities. Dose-limiting toxicity (DLT) was evaluated in the first course and included any grade≥3 non-hematological toxicities not resulting from active leukemia except alopecia; line associated venous thrombosis; infection not resulting from unexpectedly complicated myelosuppression; grade 3 fatigue, anorexia, constipation; grade 3 nausea or vomiting not requiring tube feedings, total parental nutrition or hospitalization; or grade 3 or higher transaminases that resolve to grade 1 or baseline within 5 days. Hematological DLTs included failure to recover neutrophil count (ANC>500/μL) by day 32 in patients with <5% blasts in the bone marrow, absence of myelodysplastic changes, and/or absence of evidence of disease by flow cytometry in the bone marrow. For patients with >5% blasts, these were not considered DLT. Grade 4 thrombocytopenia with clinically significant bleeding was also considered a DLT. The maximum tolerated dose (MTD) was defined as the dose level at which one or fewer of six patients in a dose level experienced DLT.

Patient characteristics. Between December 2014 and January 2017 five patients were enrolled in cohort A and eight patients were enrolled in cohort B. Eight patients (62%) had newly diagnosed untreated AML and five patients (38%) had relapsed/refractory AML. Assessment of clinical response was made according to the International Working Group criteria as published in Cheson, B. D., et al. J Clin Oncol 21, 4642-4649 (2003). Bone marrow response to treatment was evaluated by morphologic and flow cytometry studies.

Mutation analysis. Analysis of mutations in FLT3 exons 17 and 20 was conducted by deep amplicon sequencing as previously described by Baker, S. D., et al. Clin Cancer Res 19, 5758-5768 (2013), with few modifications. Briefly, FLT3 exon 17 was amplified from gDNA using forward primer 5′-TGAACGCAACAGCTTATGGA3′ and reverse primer 5′-CCATGAAGCCCTGAGATT TG-3′ (SEQ ID NO: 1). FLT3 exon 20 was amplified using forward primer 5′-TTCCATCACCGGTACCTCCTA-3′ (SEQ ID NO: 2) and reverse primer 5′-CCTGAAGCTGCAGAAAAACC-3′ (SEQ ID NO: 3). PCR amplicons were cleaned up using QIAquick PCR Purification Kit (Qiagen) and DNA concentrations were determined using an Invitrogen Quibit 3 Fluorometer and Qubit dsDNA BR Assay Kit. 0.1 ng input DNA from each exon was fragmented, tagged with adapters, and libraries were prepared using the Nextera XT DNA Sample Preparation Kit following the manufacturer's instructions (Illumina). Libraries were normalized and pooled using manufacturer's protocol then sequenced on an Illumina MiSeq System (Illumina) with 150 bp paired-end reads. Image analyses and base calling were conducted using MiSeq Control Software versions 1.5.15.1 or 2.2 and Real Time Analysis versions 1.13.148 or 1.17.28. After removing the adapter sequences, high-quality reads (Phred-like score Q30 or greater) with at least 50 nucleotides were aligned to human FLT3 reference sequence (UCSC hg19). Using CLC Genomics Workbench v11 (CLCBio) Mutations were detected and the frequencies of mutation were determined using Integrative genomics (IVG; Broad Institute). The minimum frequency of mutation detection at each genomic location was set with a threshold at the upper limit of the 99.9% confidence interval from the maximum sequencing error rate representing the platform sensitivities for detecting the low frequency allele with corresponding substitution. Table 3 shows the FLT3 exon 17 mutations detected, and Table 4 shows the FLT3 exon 20 mutations detected.

TABLE 3
List of mutations in FLT3 exon 17 by deep amplicon sequencing.

| | FLT3 Exon 17 | | | | |
|---|---|---|---|---|---|
| | | | | F691L | F691L |
| | | F691L | F691L | (TTT) | (TTT) |
| | F691 WT | (TTT) | (TTT) | C (F > L) | G/A (F > L) |
| Pt | (TTT) | C (F > L) | G/A (F > L) | (%) | (%) |
| 1* | 469569 | 51 | 73 | 0.011 | 0.015 |
| 2 | 485142 | 51 | 61 | 0.011 | 0.012 |
| 3 | 563490 | 94 | 112 | 0.017 | 0.020 |
| 4* | 560214 | 51 | 68 | 0.009 | 0.012 |
| 5 | 493229 | 44 | 76 | 0.009 | 0.015 |
| 6 | 578245 | 69 | 97 | 0.012 | 0.017 |
| 7 | 557796 | 56 | 95 | 0.010 | 0.017 |
| 8 | 550743 | 69 | 86 | 0.013 | 0.015 |
| 9 | 511748 | 89 | 167 | 0.017 | 0.032 |
| 10 | 524412 | 42 | 70 | 0.008 | 0.013 |
| 11 | 510672 | 95 | 116 | 0.019 | 0.023 |
| 12 | 555086 | 74 | 115 | 0.013 | 0.021 |
| 13 | 588343 | 88 | 154 | 0.015 | 0.026 |

*Sequencing performed on sample taken at the timepoint C1D5. All other samples were taken at the time of screening.

TABLE 4
List of mutations in FLT3 exon 20 by deep amplicon sequencing

| | FLT3 Exon 17 | | | | | | | | |
|---|---|---|---|---|---|---|---|---|---|
| | D835 WT | D835Y | D835H | D835N | D835V | D835Y | D835H | D835N | D835V |
| Pt | (GAT) | (TAT) | (CAT) | (AAT) | (GTT) | (%) | (%) | (%) | (%) |
| 1* | 806999 | 148 | 59 | 129 | 159 | 0.02 | 0.01 | 0.02 | 0.02 |
| 2 | 730394 | 135 | 41 | 129 | 136 | 0.02 | 0.01 | 0.02 | 0.02 |
| 3 | 770481 | 219 | 76 | 166 | 220 | 0.03 | 0.01 | 0.02 | 0.03 |
| 4* | 798014 | 150 | 44 | 96 | 120 | 0.02 | 0.01 | 0.01 | 0.02 |
| 5 | 801057 | 283 | 41 | 101 | 136 | 0.04 | 0.01 | 0.01 | 0.02 |
| 6 | 782173 | 169 | 51 | 122 | 152 | 0.02 | 0.01 | 0.02 | 0.02 |
| 7 | 786839 | 175 | 59 | 118 | 155 | 0.02 | 0.01 | 0.01 | 0.02 |
| 8 | 722458 | 173 | 67 | 421 | 154 | 0.02 | 0.01 | 0.06 | 0.02 |
| 9 | 777473 | 17952 | 104 | 208 | 247 | 2.3 | 0.01 | 0.03 | 0.03 |
| 10 | 739752 | 148 | 44 | 100 | 139 | 0.02 | 0.01 | 0.01 | 0.02 |
| 11 | 738506 | 272 | 108 | 166 | 237 | 0.04 | 0.01 | 0.02 | 0.03 |
| 12 | 759971 | 293 | 112 | 230 | 237 | 0.04 | 0.01 | 0.03 | 0.03 |
| 13 | 717111 | 276 | 102 | 234 | 287 | 0.04 | 0.01 | 0.03 | 0.04 |

*Sequencing performed on sample taken at the timepoint C1D5. All other samples were taken at the time of screening.

All patients were positive for a FLT3-ITD mutation. One patient (patient 9) had a baseline FLT3 D835Y mutation (VAF, 2.3%) (see Table 4).

Additional mutations co-occurring with FLT3-ITD were determined using a targeted gene panel in the OSU Department of Pathology. Genomic DNA isolated from blood leukocytes or bone marrow or tissue was profiled using digital droplet PCR method (Thunderbolt panel, Raindance Technologies) using the MiSeq Illumina platform. Analysis of the neoplasm-associated variants in the 49 genes included utilized hg19, NextGENe software and the GenomOncology platform, with a pathologist interpretation. Other co-occurring mutations were present at baseline, and these include NPM1, IDH2, and TET2 as well as other mutations (Table 5).

TABLE 5
Co-occurring mutations.

| | | % Blast (PB) | | % Blast (BM) | |
|---|---|---|---|---|---|
| Pt | Other mutations (VAF) | Baseline | C1D5 | Baseline | C1D5 |
| 1 | | 0 | 9 | 2 | NA |
| 2 | RUNX1 (0.25), TET2 (0.29) | 17 | NA | 35 | NA |
| 3 | NPM1 (0.23), WT1 (0.26), | 32.2 | 6.8 | 45 | NA |
| | DNMT3A (0.26) | | | | |
| 4 | WT1 (0.32), TET2 (0.48) | 0 | 10.4 | NA | NA |
| 5 | NPM1 (0.43), IDH2 (0.44) | 39 | 30 | 31 | NA |
| 6 | ASXL1 (0.46) | 52 | 71 | 80 | NA |
| 7 | NPM1 (0.40), TET2 (T965fs, | 69 | 65 | NA | NA |
| | 0.416; Y1245fs, 0.45) | | | | |
| 8 | IDH2 (0.50) | 35.9 | 29.3 | 65 | NA |
| 9 | NPM1 (0.41) | 49.7 | 15 | 58 | NA |
| 10 | | 88 | 20 | 88 | NA |
| 11 | NPM1 (0.43), DNMT3A | 10 | 0 | NA | NA |
| | (0.46) | | | | |
| 12 | | 0 | NA | NA | NA |
| 13 | CBL (0.34) | 14 | NA | 19.7 | NA |

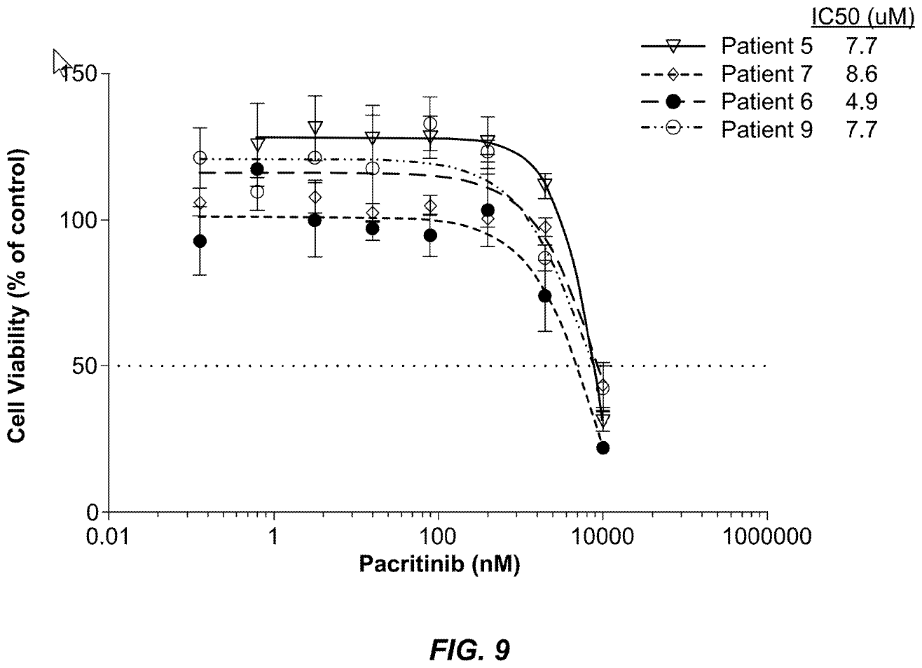

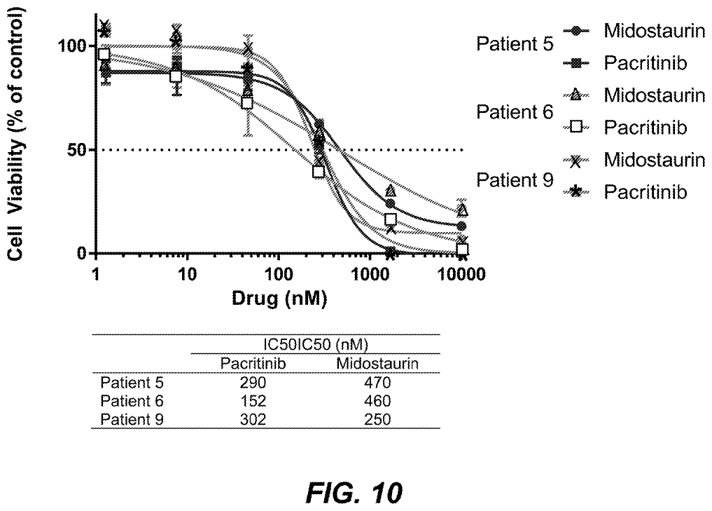

Next, the ex vivo activity of pacritinib was tested on primary blast samples from three patients in the study. Pretreated bone marrow blast samples collected during screening were treated with DMSO or increasing concentrations of pacritinib or midostaurin in RPMI supplemented with 10% FBS, hFLT3 ligand (10 ng/mL), hIL3 (10 ng/mL), hGM-CSF (10 ng/mL), and hSCF (10 ng/mL) from PeproTech for 72 hours. Viability was monitored by trypan blue in an untreated well every 24 hours. The dose response was measured at 72 hours or when viability reached 50% of less, whichever came earlier. The blasts viability to dose response was measured using CellTiter-Glo assay (Promega) according to the manufacturer's instructions (performed in 3-6 replicates). As seen in FIG. 9, pacritinib inhibited the ex vivo growth of primary blast samples with IC₅₀ values ranging from 1.9-8.6 μM. The activity of pacritinib was also tested in comparison to midostaurin on patient-derived primary blast samples. As shown in FIG. 10, pacritinib inhibited the viability of patient samples with IC50 values ranging from 152 to 302 nM, while midostaurin inhibited with IC50 values ranging from 250 to 470 nM in the same samples. The results demonstrate that Pacritinib has similar to slightly better activity compared to the current standard therapy midostaurin against these samples. In blast samples of patient 5 and 9 with IDH2 and ASXL1 co-occurring mutations, respectively, pacritinib had 1.6- and 3-fold lower respective IC50 values compared to that of midostaurin.

Pharmacokinetic studies. Serial blood samples for pacritinib plasma pharmacokinetic studies were obtained on days 1 and 21 at pretreatment and after pacritinib administration at 1, 2, 3, 5, 24 hours; a pre-dose sample of blood and bone marrow was obtained on day 5. Pacritinib was quantitated in plasma and bone marrow lysates using a validated LC/MS-MS assay in the Ohio State University (OSU) Comprehensive Cancer Center Pharmacoanalytical Shared Resource. Bone marrow concentrations were normalized to amount of protein from each sample based on a BCA assay (Thermo Scientific, Rockford, Ill.). It was assumed that 20% of total wet weight of human cells is protein, and the density of tissue was assumed as 1 g/mL for the calculation of bone marrow tissue concentrations of pacritinib based concentrations reported in Wisniewski, J. R., et al., J Proteome Res 14, 4005-4018 (2015).

All thirteen patients completed plasma pharmacokinetic studies and pacritinib bone marrow concentrations were measured in 4 patients. Pacritinib concentrations on days 1, 5 and 21 are summarized in Table 6.

TABLE 6
Pacritinib plasma and bone marrow concentrations.

| | | | Pacritinib (μg/mL) | | | | | |
|---|---|---|---|---|---|---|---|---|
| Pts | Chrt | Dose | D 1-5 h | D 1-24 h | D 5-pre-dose | D 5-BM | D 21-5 h | D 21-24 h |
| 4 | A | 100 mg | 3.0 | 5.5 | 5.7 | 4.1 | 6.5 | 7.4 |
| | | BID | | | | | | |
| 5 | A | 100 mg | 2.2 | 4.8 | 5.2 | 5.2 | 7.3 | 7.2 |
| | | BID | | | | | | |
| 8 | A | 100 mg | 3.7 | 7.9 | 5.0 | | | |
| | | BID | | | | | | |
| 9 | A | 100 mg | 5.3 | 11 | 11 | | 9.4 | 8.5 |
| | | BID | | | | | | |
| 13 | A | 100 mg | 2.5 | 4.8 | 5.3 | | 17 | 17 |
| | | BID | | | | | | |
| | | Mean | 3.7 | 6.8 | 6.4 | | 10 | 10 |
| | | SD | 1.1 | 2.5 | 2.3 | | 4.1 | 4.2 |
| 3 | B | 100 mg | | 3.6 | 4.5 | 4.8 | | |
| | | BID | | | | | | |
| 6 | B | 100 mg | 1.9 | 2.1 | 1.8 | 3.8 | 5.3 | 5.1 |
| | | BID | | | | | | |
| 7 | B | 100 mg | 1.8 | 4.0 | 3.1 | | 3.7 | 6.0 |
| | | BID | | | | | | |
| 10 | B | 100 mg | 7.4 | 12 | 7.5 | | 12 | 16 |
| | | BID | | | | | | |
| 11 | B | 100 mg | 7.4 | 12 | 28 | | 15 | 14 |
| | | BID | | | | | | |
| 12 | B | 100 mg | 1.4 | 6.1 | 4.6 | | 5.6 | 5.2 |
| | | BID | | | | | | |
| | | Mean | 4.0 | 6.5 | 8.3 | | 8.4 | 9.2 |
| | | SD | 3.1 | 4.2 | 10 | | 4.9 | 5.3 |
| All | | Mean | 3.7 | 6.7 | 7.4 | | 9.1 | 9.6 |
| 100 mg BID | | | | | | | | |
| | | SD | 2.3 | 3.5 | 7.2 | | 9.6 | 4.7 |
| 1 | B | 200 mg | 3.3 | 4.8 | 3.5 | | | |
| | | BID | | | | | | |
| 2 | B | 200 mg | 4.6 | 7.0 | 9.0 | | | |
| | | BID | | | | | | |

On day 1, mean±standard deviation plasma concentrations were similar at the 100 mg twice daily dose level in both cohorts A and B at 5 h (3.7±1.1 μg/mL and 4.0±3.1 μg/mL, respectively) and 24 h (6.8±2.5 μg/mL and 6.5 μg/mL, respectively) after drug administration. Maximum steady-state concentrations were reached on day 21 (9.6±4.7 μg/mL in both cohorts A and B combined). Pacritinib concentrations in bone marrow samples were similar to corresponding plasma concentrations.

Pacritinib plasma concentrations achieved at 100 mg twice daily, regardless of treatment cohort, were similar to those who received 200 mg once daily or twice daily in a cohorts of patients with myelofibrosis as reported in Al-Fayoumi, S., et al. EHA (2017); and Verstovsek, S., et al. Journal of hematology & oncology 9, 137 (2016). This suggests that further dose escalation above 100 mg BID may not be necessary in FLT3-ITD+ AML. Bone marrow concentrations of pacritinib were measured in two patients from each cohort. Pacritinib exposure in bone marrow was similar to that of plasma in each patient indicating pacritinib was well accumulated at the site of action.

In total, two patients in Cohort A achieved CR and one patient in cohort B achieved MLFS. The two patients who attained CR were able to receive allogeneic stem cell transplants. One of these two patients (patient 9) had a minor baseline FLT3 TKD D835Y clone. While it is possible that the two patients in cohort A who achieved CR would have done so with standard 7+3 induction alone, it is notable that the percentage of peripheral blood blasts in patient 9 was reduced by 35% after the first 5 days of pacritinib therapy without receiving cytarabine and daunorubicin.

Many patients had other co-occurring mutations at baseline. Both patient 5 and 9 who achieved CR had NPM1 mutations, which is associated with a better prognosis in FLT3-ITD+ AML. However, patient 5 who also had a IDH2 mutation did not respond to pacritinib single therapy in first 5 days as patient 9 did who had the NPM1 mutation alone indicated by their blast count reduction from pre-treatment to day 5 of therapy.

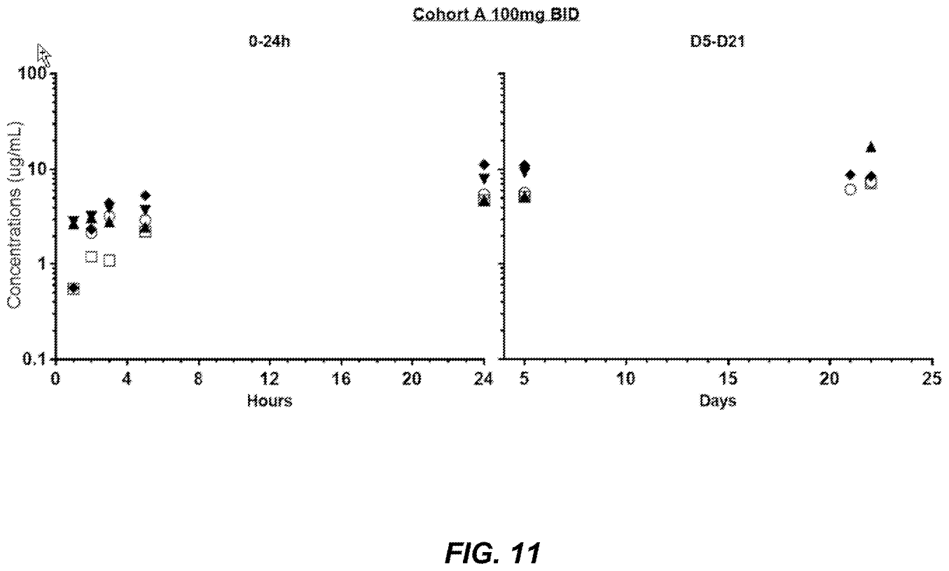

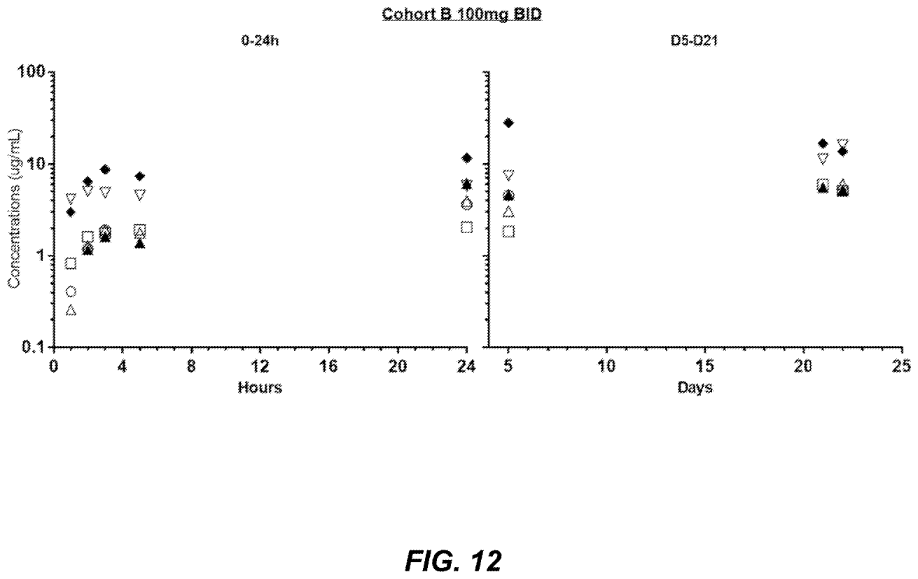

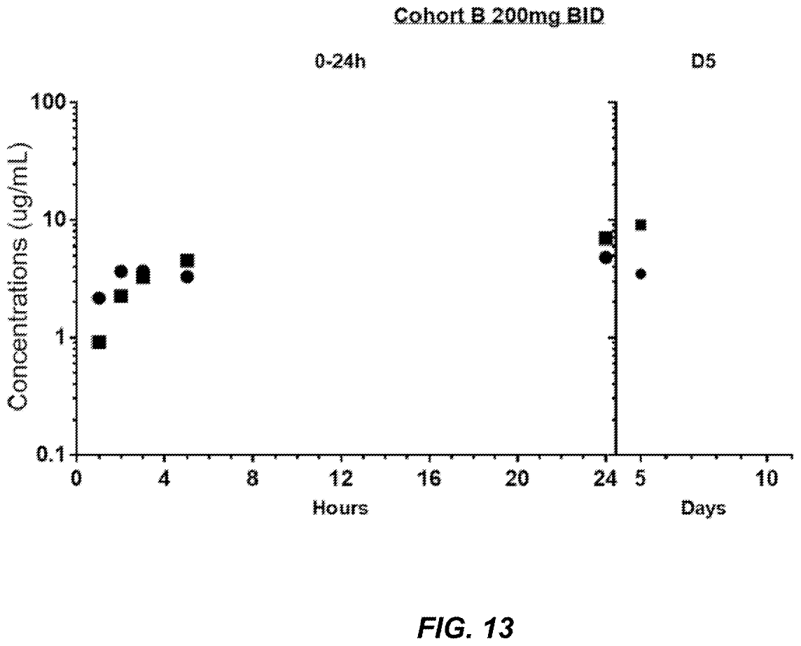

Of thirteen patients, all patients were evaluable for toxicity, and ten patients were evaluable for response. One patient in cohort A was not evaluable for treatment response due to early death and 2 patients in cohort B were not evaluable for response due to early discontinuation of therapy. Of four evaluable patients in cohort A, two patients (5 and 9) achieved complete remission, and two patients had stable disease. In cohort B, two patients had stable disease, and one patient achieved morphologic leukemia free-state. One patient that had a minor clone with a FLT3 D835Y (3%) mutation at baseline achieved complete remission. FIG. 11 shows the clinical PK profiles for pacritinib in plasma from the five cohort A patients who received 100 mg twice daily. FIG. 12 shows the clinical PK profiles for pacritinib in plasma from the six cohort B patients who received 100 mg twice daily. FIG. 13 shows the clinical PK profiles for pacritinib in plasma from the two cohort B patients who received 200 mg twice daily. The pacritinib plasma exposure parameters (Cmax and Cmin) are summarized in Table 3 below.

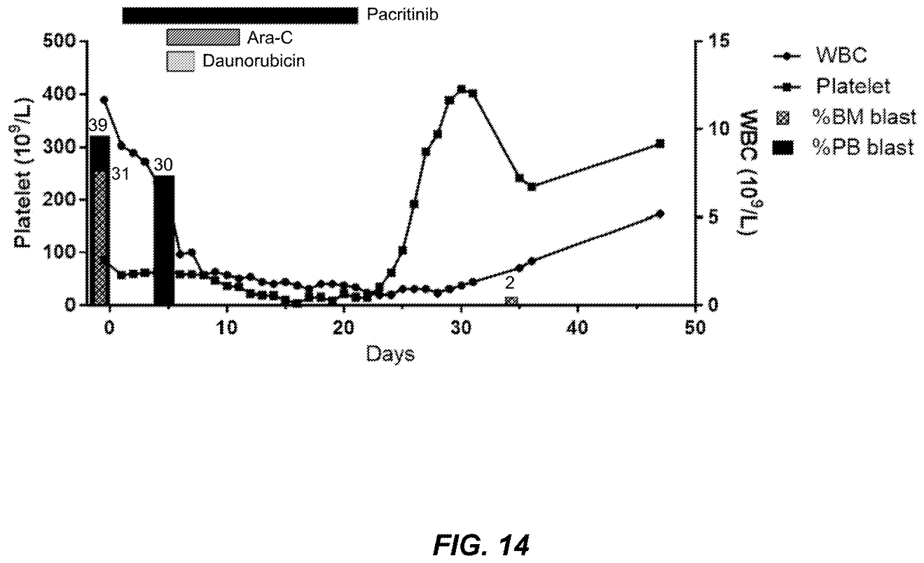

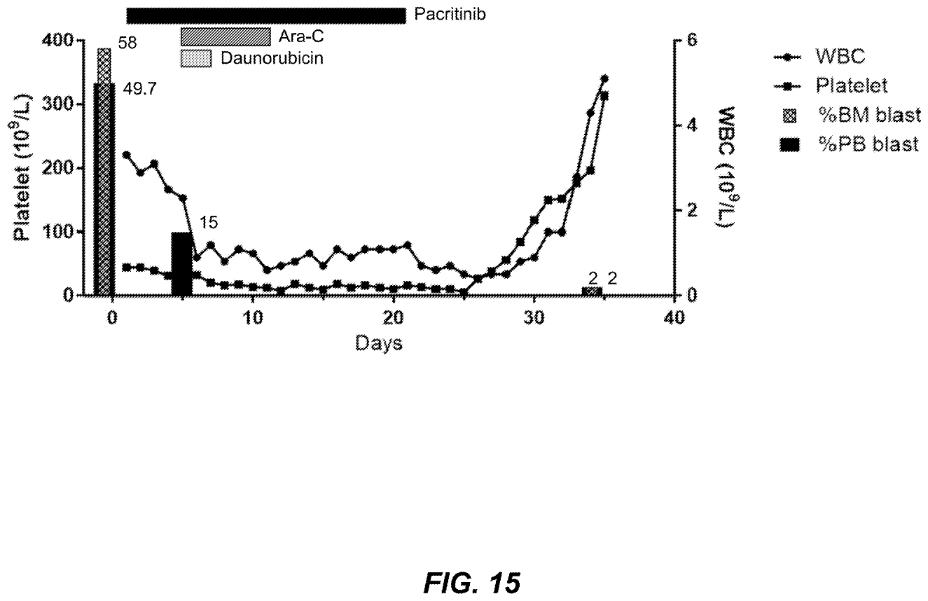

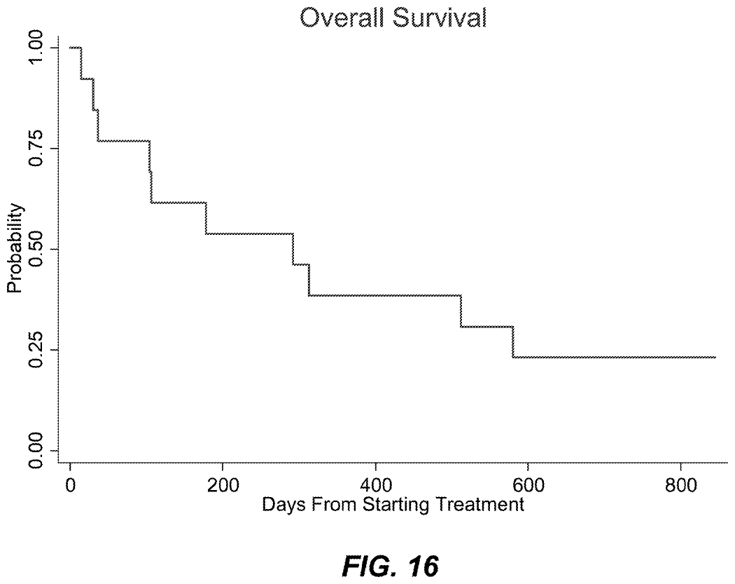

FIG. 14 shows the clinical course of patient 9 and FIG. 15 shows the clinical course of patient 9. Patient 9, whom achieved a CR, had a minor clone with a FLT3 D835Y mutation at baseline. In cohort B, one patient achieved morphologic leukemia free state, and five patients had stable disease. Median survival in both cohorts was 292 days (95% CI: 36-580), and response rate defined as CR or morphologic leukemia free state (MLFS) was 23.1% (95% CI: 5.0-53.8%). Kaplan-Meier analysis for overall survival of patients who received pacritinib is shown in FIG. 16.

In conclusion, pacritinib was relatively well tolerated at a dose of 100 mg twice daily in combination with intensive or non-intensive chemotherapy and demonstrated preliminary activity in patients newly diagnosed and relapsed/refractory FLT3-ITD+ AML.

All of the U.S. patents, U.S. patent application publications, U.S. patent applications, foreign patents, foreign patent applications and non-patent publications referred to in this specification or the attached Application Data Sheet, including but not limited to U.S. Provisional Application Ser. No. 62/739,802 filed Oct. 1, 2018, and U.S. Provisional Application Ser. No. 62/838,239 filed Apr. 24, 2019, are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating a hematologic cancer, the method comprising:
administering an effective amount of a therapeutic agent having inhibitory activity against Janus kinase 2 (JAK2) and fms-like tyrosine kinase 3 (FLT3) to a subject having a predetermined genetic profile comprising an FLT3 mutation.

2. The method of claim 1, wherein the FLT3 mutation comprises an internal tandem duplication (ITD) mutation.

3. The method of claim 1, wherein the FLT3 mutation comprises a tyrosine kinase domain (TKD) mutation.

4. The method of claim 3, wherein the TKD mutation is a FLT 835 mutation.

5-6. (canceled)

7. The method of claim 3, wherein the TKD mutation comprises an FLT3 691 mutation.

8-9. (canceled)

10. The method of claim 1, wherein the FLT3 mutation comprises ITD-835H, ITD-835V, ITD-D835Y, or ITD-F691L.

11. (canceled)

12. The method of claim 1, wherein the therapeutic agent is pacritinib, or a pharmaceutically acceptable salt, prodrug, or N-oxide thereof.

13-15. (canceled)

16. The method of claim 1, wherein the method further comprises administering an effective amount of a nucleoside analog, an intercalating agent, a hypomethylating agent, or combinations thereof.

17-18. (canceled)

19. The method of claim 16, wherein the nucleoside analog comprises cytarabine.

20. The method of claim 16, wherein the intercalating agent comprises daunorubicin.

21. (canceled)

22. The method of claim 16, wherein the hypomethylating agent comprises decitabine or azacytidine.

23. The method of claim 1, wherein the hematologic cancer comprises acute myeloid leukemia.

24. (canceled)

25. A method for treating acute myeloid leukemia, the method comprising:
administering an effective amount of pacritinib, or a pharmaceutically acceptable salt, prodrug, or N-oxide thereof to a subject having a predetermined mutation in FLT3, the mutation comprising:
i. an internal tandem duplication (ITD) mutation; and/or
ii. a tyrosine kinase domain (TKD) mutation.

26. The method of claim 25, wherein the FLT3 mutation comprises an ITD mutation and a TKD mutation.

27-36. (canceled)

37. The method of claim 25, wherein the method further comprises administering an effective amount of a nucleoside analog, an intercalating agent, a hypomethylating agent, or combinations thereof.

38-43. (canceled)

44. The method of claim 25, wherein the acute myeloid leukemia is relapsed or refractory acute myeloid leukemia.

45. A method of selecting a treatment regimen for a subject in need of treatment for a hematologic cancer, the method comprising:
receiving a genetic profile for the subject, the genetic profile comprising an FLT3 mutation; and
selecting a treatment regimen based on the genetic profile, the treatment regimen comprising a therapeutic agent having inhibitory activity against Janus kinase 2 (JAK2) and fms-like tyrosine kinase 3 (FLT3).

46. (canceled)

47. The method of claim 45, wherein the FLT3 mutation is a tyrosine kinase domain (TKD) mutation.

48-55. (canceled)

56. The method of claim 45, wherein the therapeutic agent is pacritinib, or a pharmaceutically acceptable salt, prodrug, or N-oxide thereof.

57-61. (canceled)

62. The method of claim 45, wherein the treatment regimen further comprises administering a nucleoside analog, an intercalating agent, a hypomethylating agent, or combinations thereof.

63-68. (canceled)

69. The method of claim 45, wherein the hematologic cancer comprises acute myeloid leukemia.

70-80. (canceled)
